(19) United States
(12) Patent Application Publication
(10) Pub. No.: US 2022/0228104 A1
(43) Pub. Date: Jul. 21, 2022

(54) ENGINEERED ORGANISMS AND USES THEREOF AS LIVING MEDICINES, RESEARCH TOOLS, FOOD PRODUCTS, OR ENVIRONMENTAL TOOLS

(71) Applicant: 64-X, Inc., San Francisco, CA (US)

(72) Inventors: Ryan Gallagher, San Francisco, CA (US); Alexis Rovner, San Francisco, CA (US); George Church, Brookline, MA (US); Jeffrey Way, San Francisco, CA (US); Pamela Silver, San Francisco, CA (US)

(21) Appl. No.: 17/611,005

(22) PCT Filed: May 14, 2020
(86) PCT No.: PCT/US20/33004

§ 371 Date: Nov. 12, 2021

Related U.S. Application Data
(60) Provisional application No. 62/847,904, filed on May 14, 2019.
(60) Provisional application No. 62/847,928, filed on May 14, 2019.
(60) Provisional application No. 62/847,936, filed on May 14, 2019.
(60) Provisional application No. 62/847,910, filed on May 14, 2019.

(51) Int. Cl. C12N 1/20; G16B 5/00; G16B 40/20; C12N 15/70; C12N 15/90

(52) U.S. Cl. CPC C12N 1/20; G16B 5/00; C12N 2800/101; C12N 15/70; C12N 15/902; G16B 40/20

(57) ABSTRACT

Provided herein are engineered organism containing a transgene in which expression of the transgene in an open environment is prevented or reduced, for example, by recoding designs. Also provided are methods of producing such engineered organism and use of such engineered organisms as therapeutics or for producing food, food supplement, and animal feed products.

### CROSS-REFERENCE TO RELATED APPLICATIONS

This application claims the benefit of and priority to U.S. Provisional Patent Application No. 62/847,904, filed May 14, 2019; U.S. Provisional Patent Application No. 62/847,928, filed May 14, 2019; U.S. Provisional Patent Application No. 62/847,910, filed May 14, 2019; and U.S. Provisional Patent Application No. 62/847,936, filed May 14, 2019, the disclosure of each of which is hereby incorporated by reference in its entirety for all purposes.

### TECHNICAL FIELD OF THE INVENTION

This invention is related to methods of generating engineered organisms with targeted genome designs and targeted functional properties. The invention also relates to methods of generating released engineered organisms that produce biomanufactured products, such as nucleic acids, polypeptides, their monomers (nucleotides and amino acids), small molecules, and metabolites. The invention also relates to uses of released engineered organisms as medicines (e.g., living therapeutics, living vaccines), research tools (e.g., use of living therapeutics or living vaccines for research or diagnostic use), food products e.g, probiotics, ingredients), and environmental tools (e.g., bioremediation). In particular, it relates to released engineered organisms that are enhanced for the production of these products and optimized for these applications.

### BACKGROUND OF THE INVENTION

Expanding markets include those where bacterial organisms are engineered to produce biomanufactured products such as nucleic acids, polypeptides, their monomers, small molecules, and metabolites, and then released into open environments. For example, these markets can include engineered bacterial organisms that are used as: medicines (e.g., living therapeutics, living vaccines), research tools (e.g., use of living therapeutics or living vaccines for research or diagnostic use), food products (e.g, probiotics, ingredients), or environmental tools (e.g., bioremediation).

There is a continuing need in the art for next generation engineered organisms that are enhanced in their ability to produce biomanufactured products, and that are optimized (e.g., horizontal gene transfer resistant) for release into these open environments, to enable increasingly advanced applications, many of which have yet to come to market.

There is also a continuing need in the art for methods of producing these advanced engineered organisms using processes that are more time-effective, cost-effective and scalable, using current good manufacturing practices (cGMP) or non-cGMP conditions.

### SUMMARY OF THE INVENTION

In one aspect, the present disclosure provides a genetically engineered released bacterial organism comprising engineered genetic material,

the material comprising:

i. a) at least one genetically engineered codon and b) at least one genetically engineered endogenous element, and

ii. at least one exogenous nucleic acid sequence encoding a therapeutic polypeptide or portion thereof,

wherein the at least one genetically engineered naturally occurring element comprises a modification to or deletion of (a) a first nucleic acid sequence encoding a transfer RNA cognate to the genetically engineered codon and optionally (b) a second nucleic acid sequence encoding a release factor cognate to a second genetically engineered second codon.

and wherein the released bacterial organism is capable of producing the therapeutic polypeptide or portion thereof.

In certain embodiments, the at least one genetically engineered codon is present within the bacterial genome. In certain embodiments, the at least one genetically engineered codon is present outside the bacterial genome. In certain embodiments, the at least one genetically engineered endogenous element is present within the bacterial genome. In certain embodiments, the at least one genetically engineered endogenous element is present outside the bacterial genome. In certain embodiments, the at least one exogenous nucleic acid sequence is present within the bacterial genome. in certain embodiments, the at least one exogenous nucleic acid sequence is present outside the bacterial genome.

In certain embodiments, the engineered genetic material comprises at least one heterologous nucleic acid sequence. In certain embodiments, the engineered genetic material comprises from at least two to over 100 heterologous nucleic acid sequences. In certain embodiments, the engineered genetic material comprises from at least two to over 100 genetically engineered endogenous elements. In certain embodiments, the engineered genetic material comprises synthetic nucleic acid sequences.

In certain embodiments, the bacteria comprise Escherichia coli, Escherichia coli NGF-1, Escherichia coli UU2685, Escherichia coli K-12 MG1655, Escherichia coli “recoded” or “GRO” strains and derivatives, Escherichia coli C7 strains, Escherichia coli C7□A strains, Escherichia cob C13 strains, Escherichia coli C13□A strains, Escherichia coli “C321 strains”, Escherichia coli C321□A strains, Escherichia coli C321□A “synthetic auxotroph” strains and derivatives, Escherichia coli evolved C321 strains, Escherichia coli C321.ΔA.M9adapted strains, Escherichia coli C321.ΔA.opt strains, Escherichia cob rE.coli-57 strains and derivatives, Escherichia coli C321□A “Syn61” strains and derivatives, Escherichia coli K-12 MG1655 “MDS” strains and derivatives, Escherichia coli K-12 MG1655 MDS9 strains, Escherichia coli K-12 MG1655 MDS12 strains, Escherichia coli K-12 MG1655 MDS41 strains, Escherichia coli K-12 MG1655 MDS42 strains, Escherichia coli K-12 MG1655 MDS43 strains, Escherichia coli K-12 MG1655 MDS66 strains, Escherichia coli BL21 DE3, Escherichia coli BL21 hybrid strains (“BLK strains”), Escherichia coli Nissle 1917, Salmonella, Salmonella typhimurium, Salmonella typhi Ty21a, Lactobacillus, Lactobacillus plantarum, Lactobacillus reuteri, Lactobacillus gasseri, Lactobacillus gasseri BNR17, Lactobacillus fermentum KLD, Lactobacillus helveticus, Lactobacillus helveticus strain NS8, Lactococcus, Lactococcus lactis, Lactococcus lactis NZ9000, Lactococcus NZ3900, Lactococcus lactis NZ9001, Lactococcus lactis MG1363, Bacteroides, Bacteroides thetaiotaomicron, Bacteroides fragilis, Bacteroides vulgatus, Bacteroides ovatus, Bacteroides uniformis, Bacteroides eggerthii, Bacteroides xylanisolvens, Bacteroides intestinalis, Bacteroides dorei, Bacteroides cellulosilyticus, Bacillus, Bacillus subtilis, Acetobacter, Streptomyces, Streptococcus, Staphylococcus, Staphylococcus epidermis, Bifidobacterium, Bifidobacterium longum, Bifidobacterium infantis, Eubacterium, Corynebacterium, Corynebacterium glutamicum, Rumunococcus, Coprococcus, Fusobacterium, Clostridium, Clostridium butyricum, Shewanella, Cyanobacterium, Mycoplasma, Mycoplasma capricolum, Mycoplasma genitalium, Mycoplasma mycoides, Mycoplasma mycoides JCVI-syn strains, Mycoplasma mycoides JCVI-syn3.0 strains, Listeria, Listeria monocytogenes, Vibrio, Vibrio cholerae, Vibrio natriegens, Vibrio natriegens Vmax strains, Pseudomonas and variants and progeny thereof.

In certain embodiments, the at least one genetically engineered codon comprises at least one recoded codon. In certain embodiments, the at least one genetically engineered codon comprises between two and seven recoded codons. In certain embodiments, the at least one genetically engineered codon comprises at least one recoded stop codon. In certain embodiments, the at least one genetically engineered codon comprises at least one recoded sense codon. In certain embodiments, the recoded codon comprises a sense codon, and wherein the recoded codon is synonymously replaced in the engineered genetic material. In certain embodiments, the recoded codon comprises a stop codon, and wherein the recoded codon is synonymously replaced in the engineered genetic material.

In certain embodiments, the engineered genetic material comprises a plurality of recoded codons, wherein the recoded codons comprise (i) a sense codon and (ii) a stop codon, and wherein at least one of (i) and (ii) is synonymously replaced in the engineered genetic material. In certain embodiments, the engineered genetic material comprises two to seven recoded codons, wherein the recoded codons comprise (i) a sense codon and (ii) a stop codon, and wherein at least one of (i) and (ii) is synonymously replaced in the engineered genetic material. In certain embodiments, the engineered genetic material comprises replacement of all instances of at least stop codon and at least one sense codon with a second codon in all essential genes. In certain embodiments, the engineered genetic material comprises replacement of all instances of at least stop codon and at least one sense codon with a second codon in all genes essential for viability of the genetically engineered bacterial organism. In certain embodiments, the engineered genetic material comprises replacement of all instances of at least stop codon with a second codon in all genes essential for viability of the genetically engineered bacterial organism. In certain embodiments, the engineered genetic material comprises replacement of all instances of at least one sense codon with a second codon in all genes essential for viability of the genetically engineered bacterial organism. In certain embodiments, the engineered genetic material comprises replacement of all instances of at least stop codon and at least one sense codon with a second codon in all genes essential for bacterial fitness of the genetically engineered bacterial organism. In certain embodiments, the engineered genetic material comprises replacement of all instances of at least stop codon with a second codon in all genes essential for bacterial fitness of the genetically engineered bacterial organism. In certain embodiments, the engineered genetic material comprises replacement of all instances of at least one sense codon with a second codon in all genes essential for bacterial fitness of the genetically engineered bacterial organism. In certain embodiments, the engineered genetic material comprises replacement of all instances of at least stop codon and at least one sense codon with a second codon in all genes essential for bacterial homeostasis of the genetically engineered bacterial organism. In certain embodiments, the engineered genetic material comprises replacement of all instances of at least stop codon with a second codon in all genes essential for bacterial homeostasis of the genetically engineered bacterial organism. In certain embodiments, the engineered genetic material comprises replacement of all instances of at least one sense codon with a second codon in all genes essential for bacterial homeostasis of the genetically engineered bacterial organism. In certain embodiments, the recoded codon comprises a sense codon, and wherein the recoded codon is synonymously replaced in from less than 1% to at least about 99% of the engineered genetic material. In certain embodiments, the recoded codon comprises a stop codon, and wherein recoded codon is synonymously replaced in from less than 1% to at least about 99% of the engineered genetic material. In certain embodiments, the genetically engineered released bacterial organism comprises a plurality of recoded codons, wherein the recoded codons comprise (i) at least one sense codon and (ii) at least one stop codon, and wherein at least one of (i) and (ii) is synonymously replaced in from less than 1% to at least about 99% of the engineered genetic material.

In certain embodiments, the engineered genetic material further comprises at least one orthogonal translation system (OTS) comprising an aminoacyl-tRNA synthetase (aaRS) and cognate tRNA, and wherein the tRNA of the at least one OTS comprises an anticodon complementary to a recoded codon. In certain embodiments, the engineered genetic material further comprises at least one orthogonal translation system (OTS) comprising an aminoacyl-tRNA synthetase (aaRS) and cognate tRNA, wherein time tRNA of the at least one OTS comprises an anticodon complementary to a recoded codon, and wherein the tRNA charges a synthetic or unnatural amino acid. In certain embodiments, the engineered genetic material further comprises at least one orthogonal translation system (OTS) comprising an aminoacyl-tRNA synthetase (aaRS) and cognate tRNA, wherein the tRNA of the at least one OTS comprises an anticodon complementary to a recoded codon, and wherein the tRNA charges a natural amino acid. In certain embodiments, the engineered genetic material further comprises at least one suppressor tRNA, wherein the tRNA of time at least one suppressor tRNA comprises an anticodon complementary to a recoded codon, and wherein the tRNA charges a natural amino acid. In certain embodiments, the engineered genetic material further comprises a deletion or modification to at least one phage receptor gene or portion thereof. In certain embodiments, the engineered genetic material does not comprise a deletion or modification to at least one phage receptor gene or portion thereof.

In another aspect, the present disclosure provides a population comprising a plurality of the genetically engineered released bacterial organism of claim 1, wherein the population is capable of continuously sustaining cGMP manufacturing of the therapeutic polypeptide.

In certain embodiments, the population is capable of continuously sustaining cGMP manufacturing of the therapeutic polypeptide in the presence of a phage population. In certain embodiments, the population is capable of continuously sustaining cGMP manufacturing of the therapeutic polypeptide in the presence of an unknown phage population. In certain embodiments, the population has a higher viral resistance capacity compared to a reference bacterial population that comprises the exogenous nucleic acid sequence but does not comprise the at least one genetically engineered codon, and wherein the population is suitable for cGMP manufacturing of the therapeutic polypeptide or a nucleic acid encoding the therapeutic polypeptide.

In certain embodiments, the viral resistance capacity allows the population to continuously sustain cGMP manufacturing of the therapeutic polypeptide or a nucleic acid encoding the therapeutic polypeptide in the presence of an unidentified phage population at least about 10% longer than continuously sustained cGMP manufacturing of the therapeutic polypeptide or the nucleic acid encoding the therapeutic polypeptide using the reference bacterial population. In certain embodiments, the viral resistance capacity allows the population to continuously sustain cGMP manufacturing of the therapeutic polypeptide or a nucleic acid encoding the therapeutic polypeptide at least about 10% longer than continuously sustained cGMP manufacturing of the therapeutic polypeptide or the nucleic acid encoding the therapeutic polypeptide using the reference bacterial population. In certain embodiments, the viral resistance capacity allows the population to continuously sustain cGMP manufacturing of the therapeutic polypeptide or a nucleic acid encoding the therapeutic polypeptide from at least about 10% longer to greater than 100% longer than continuously sustained cGMP manufacturing of the therapeutic polypeptide or the nucleic acid encoding the therapeutic polypeptide using the reference bacterial population. In certain embodiments, the viral resistance capacity allows the population to continuously sustain cGMP manufacturing of the therapeutic polypeptide or the nucleic acid encoding the therapeutic polypeptide for greater than 1, 2, 3, 4, 5, 6 or 7 days, or greater than 1, 2, 3, 4 weeks. In certain embodiments, the population has a cGMP manufacturing productivity over a given period of time compared to a reference bacterial population that comprises the exogenous nucleic acid sequence but does not comprise the at least on engineered codon.

In another aspect, the present disclosure provides a genetically engineered released bacterial organism comprising engineered genetic material, the material comprising:

i. a plurality of genetic modifications comprising replacement of all instances of at least one type of first codon with a second codon in all essential genes,

ii. at least one genetically engineered endogenous element, and

iii. at least one exogenous nucleic acid sequence encoding a therapeutic polypeptide or portion thereof,

wherein the at least one genetically engineered endogenous element comprises a modification to or deletion of: (a) a nucleic acid sequence encoding a transfer RNA that recognizes the at least one type of first codon, (b) a nucleic acid sequence encoding a release factor that recognizes the at least one type of first codon, or (c) a combination of (a) and (b) in the same genetically engineered bacterial organism, and

and wherein the released bacterial organism is capable of producing the therapeutic polypeptide or portion thereof.

In another aspect, the present disclosure provides a genetically engineered released bacterial organism comprising engineered genetic material,

the material comprising:

a) at least one genetically engineered codon and b) at least one genetically engineered endogenous element, wherein the at least one genetically engineered endogenous element comprises a modification to or deletion of (a) a first nucleic acid sequence encoding a transfer RNA and optionally (b) a second nucleic acid sequence encoding a release factor,

wherein the released bacterial organism is capable of producing a polypeptide or portion thereof or a nucleic acid.

In another aspect, the present disclosure provides a genetically engineered released bacterial organism comprising engineered genetic material,

the material comprising:

i. a) at least one genetically engineered codon and b) at least one genetically engineered endogenous element, and

ii. at least one exogenous nucleic acid sequence encoding a polypeptide or portion thereof, suitable for synthesis of a therapeutic polypeptide

wherein the at least one genetically engineered naturally occurring element comprises a modification to or deletion of (a) a first nucleic acid sequence encoding a transfer RNA cognate to the genetically engineered codon and optionally (b) a second nucleic acid sequence encoding a release factor cognate to a second genetically engineered second codon.

and wherein the released bacterial organism is capable of producing the therapeutic polypeptide or portion thereof.

In another aspect, the present disclosure provides a genetically engineered released bacterial organism comprising engineered genetic material,

the material comprising:

i. a) at least one genetically engineered codon and b) at least one genetically engineered endogenous element, and

ii. at least one exogenous nucleic acid sequence encoding a polypeptide or portion thereof, suitable for synthesis of a therapeutic nucleic acid

wherein the at least one genetically engineered naturally occurring element comprises a modification to or deletion of (a) a first nucleic acid sequence encoding a transfer RNA cognate to the genetically engineered codon and optionally (b) a second nucleic acid sequence encoding a release factor cognate to a second genetically engineered second codon.

and wherein the released bacterial organism is capable of producing a polypeptide or portion thereof or a nucleic acid.

In another aspect, the present disclosure provides a genetically engineered released bacterial organism comprising engineered genetic material,

the material comprising:

i. a) at least one genetically engineered codon and b) at least one genetically engineered endogenous element, and

ii. at least one exogenous nucleic acid sequence encoding a polypeptide or portion thereof, suitable for synthesis of a therapeutic viral particle

wherein the at least one genetically engineered naturally occurring element comprises a modification to or deletion of (a) a first nucleic acid sequence encoding a transfer RNA cognate to the genetically engineered codon and optionally (b) a second nucleic acid sequence encoding a release factor cognate to a second genetically engineered second codon.

and wherein the released bacterial organism is capable of producing a polypeptide or portion thereof or a nucleic acid.

In another aspect, the present disclosure provides a genetically engineered released bacterial organism comprising engineered genetic material,

the material comprising:

i. a) at least one genetically engineered codon and b) at least one genetically engineered endogenous element, and

ii. at least one exogenous nucleic acid sequence suitable for synthesis of a therapeutic nucleic acid

wherein the at least one genetically engineered naturally occurring element comprises a modification to or deletion of (a) a first nucleic acid sequence encoding a transfer RNA cognate to the genetically engineered codon and optionally (b) a second nucleic acid sequence encoding a release factor cognate to a second genetically engineered second codon.

and wherein the released bacterial organism is capable of producing the therapeutic nucleic acid.

In another aspect, the present disclosure provides a genetically engineered released bacterial organism comprising engineered genetic material,

the material comprising:

i. a) at least one genetically engineered codon and b) at least one genetically engineered endogenous element, and

ii. at least one exogenous nucleic acid sequence encoding a polypeptide or portion thereof, wherein the polypeptide or portion thereof is contacted with a cell ex vivo,

wherein the at least one genetically engineered naturally occurring element comprises a modification to or deletion of (a) a first nucleic acid sequence encoding a transfer RNA cognate to the genetically engineered codon and optionally (b) a second nucleic acid sequence encoding a release factor cognate to a second genetically engineered second codon.

and wherein the released bacterial organism is capable of producing the polypeptide or portion thereof.

In another aspect, the present disclosure provides a genetically engineered released bacterial organism comprising engineered genetic material,

the material comprising:

i. a) at least one genetically engineered codon and b) at least one genetically engineered endogenous element, and

ii. at least one exogenous nucleic acid sequence suitable for synthesis of a nucleic acid

wherein the at least one genetically engineered naturally occurring element comprises a modification to or deletion of (a) a first nucleic acid sequence encoding a transfer RNA cognate to the genetically engineered codon and optionally (b) a second nucleic acid sequence encoding a release factor cognate to a second genetically engineered second codon.

and wherein the released bacterial organism is capable of producing the nucleic acid.

In another aspect, the present disclosure provides a genetically engineered released bacterial organism comprising engineered genetic material,

the material comprising:

i. a) at least one genetically engineered codon and b) at least one genetically engineered endogenous element, and

ii. at least one exogenous nucleic acid sequence suitable for synthesis of a therapeutic nucleic acid, wherein the therapeutic nucleic acid is contacted with a cell ex vivo

wherein the at least one genetically engineered naturally occurring element comprises a modification to or deletion of (a) a first nucleic acid sequence encoding a transfer RNA cognate to the genetically engineered codon and optionally (b) a second nucleic acid sequence encoding a release factor cognate to a second genetically engineered second codon.

and wherein the released bacterial organism is capable of producing the therapeutic nucleic acid.

In another aspect, the present disclosure provides a genetically engineered released bacterial organism comprising engineered genetic material,

the material comprising:

i. a) at least one genetically engineered codon and b) at least one genetically engineered endogenous element, and

ii. at least one exogenous nucleic acid sequence suitable for synthesis of a synthesized nucleic acid, wherein the synthesized nucleic acid is contacted with a cell ex vivo

wherein the at least one genetically engineered naturally occurring element comprises a modification to or deletion of (a) a first nucleic acid sequence encoding a transfer RNA cognate to the genetically engineered codon and optionally (b) a second nucleic acid sequence encoding a release factor cognate to a second genetically engineered second codon.

and wherein the released bacterial organism is capable of producing the synthesized nucleic acid.

In another aspect, the present disclosure provides a genetically engineered released bacterial organism comprising engineered genetic material,

the material comprising:

i. a) at least one genetically engineered codon and b) at e genetically engineered endogenous element, and

ii. at least one exogenous nucleic acid sequence encoding a polypeptide or portion thereof, suitable for synthesis of a viral particle

wherein the at least one genetically engineered naturally occurring element comprises a modification to or deletion of (a) a first nucleic acid sequence encoding a transfer RNA cognate to the genetically engineered codon and optionally (b) a second nucleic acid sequence encoding a release factor cognate to a second genetically engineered second codon.

and wherein the released bacterial organism is capable of producing the polypeptide or portion thereof.

In another aspect, the present disclosure provides a genetically engineered released bacterial organism comprising engineered genetic material,

the material comprising:

i. a) at least one genetically engineered codon and b) at least one genetically engineered endogenous element, and

at least one exogenous nucleic acid sequence encoding a polypeptide or portion thereof,

wherein the at least one genetically engineered naturally occurring element comprises a modification to or deletion of (a) a first nucleic acid sequence encoding a transfer RNA cognate to the genetically engineered codon and optionally (b) a second nucleic acid sequence encoding a release factor cognate to a second genetically engineered second codon.

and wherein the released bacterial organism is capable of producing the polypeptide or portion thereof.

In another aspect, the present disclosure provides a genetically engineered released bacterial organism comprising engineered genetic material,

the material comprising:

i. a) at least one genetically engineered codon and b) at least one genetically engineered endogenous element, and

ii. at least one exogenous nucleic acid sequence encoding a first polypeptide or portion thereof, suitable for synthesis of a second polypeptide

wherein the at least one genetically engineered naturally occurring element comprises a modification to or deletion of (a) a first nucleic acid sequence encoding a transfer RNA cognate to the genetically engineered codon and optionally (b) a second nucleic acid sequence encoding a release factor cognate to a second genetically engineered second codon.

and wherein the released bacterial organism is capable of producing the first polypeptide or portion thereof.

In another aspect, the present disclosure provides a genetically engineered released bacterial organism comprising engineered genetic material,

the material comprising:

i. a) at least one genetically engineered codon and b) at least one genetically engineered endogenous element, and

ii. at least one exogenous nucleic acid sequence encoding a polypeptide or portion thereof, suitable for synthesis of a nucleic acid

wherein the at least one genetically engineered naturally occurring element comprises a modification to or deletion of (a) a first nucleic acid sequence encoding a transfer RNA cognate to the genetically engineered codon and optionally (b) a second nucleic acid sequence encoding a release factor cognate to a second genetically engineered second codon.

and wherein the released bacterial organism is capable of producing the polypeptide or portion thereof.

In another aspect, the present disclosure provides a method of producing a plasmid, the method comprising culturing the population of genetically engineered released bacteria of any proceeding claim, under conditions such that a plasmid comprising the at least one exogenous nucleic acid sequence is produced.

In certain embodiments, the plasmid is produced under cGMP conditions. In certain embodiments, the plasmid is produced in the presence of a phage population. In certain embodiments, the population has resistance to a virus present in the culture, and wherein the culturing comprises a continuous culturing for greater than 1, 2, 3, 4, 5, 6 or 7 days, or greater than 1, 2, 3, 4 weeks.

In certain embodiments, the plasmid is capable of generating a virus selected from a lentivirus, adenovirus, herpes virus, adeno-associated virus, or a portion thereof. In certain embodiments, the plasmid is capable of generating a nucleic acid selected from a DNA or an RNA. In certain embodiments, the plasmid is capable of generating an RNA selected from a shRNA, siRNA, mRNA, linear RNA, or circular RNA.

In another aspect, the present disclosure provides a method of producing a polypeptide, the method comprising culturing the population of genetically engineered released bacteria of any proceeding claim, wherein the population comprises at least one exogenous nucleic acid sequence encoding a polypeptide or portion thereof, under conditions such that the polypeptide or portion thereof is produced.

In certain embodiments, the polypeptide or portion thereof is produced under cGMP conditions. In certain embodiments, the polypeptide or portion thereof is produced in the presence of a phage population. In certain embodiments, the population has resistance to a virus present in the culture, and wherein the culturing comprises a continuous culturing for greater than 1, 2, 3, 4, 5, 6 or 7 days, or greater than 1, 2, 3, 4 weeks. In certain embodiments, the polypeptide or portion thereof is a human or humanized polypeptide or portion thereof.

In another aspect, the present disclosure provides a method for generating a population of genetically engineered released bacteria, comprising the steps of:

i. contacting an isolated precursor bacterial strain comprising a plurality of bacteria with (i) a first plurality of nucleic acid sequences that replace a first target genome region in the precursor bacterial strain genome, and (ii) a second plurality of nucleic acid sequences that replace a second target genome region in the precursor bacterial strain genome, to produce a genetically engineered bacterium comprising a single nucleic acid sequence from each of the first plurality and the second plurality of nucleic acid sequences;

ii. culturing the genetically engineered bacterium to produce a population of genetically engineered released bacteria.

In certain embodiments, each of the first plurality and the second plurality of nucleic acid sequences comprise at least one genetically engineered endogenous element comprises a modification to or deletion of (a) a first nucleic acid sequence encoding a transfer RNA and optionally (b) a second nucleic acid sequence encoding a release factor.

### BRIEF DESCRIPTION OF THE DRAWINGS

FIG. 1—A flow chart illustrating the relationship between an entity, base strain, engineered. organism (EO), and a released engineered organism (REO).

FIG. 2—A series of chemical structures of nonstandard amino acids (NSAAs)

FIG. 3—A flow chart illustrating the relationship between an entity, base strain, recoded organism (RO), and a released recoded organism (RRO).

FIG. 4—An exemplary recoding scheme whereby two serine sense codons are recoded to two synonymous serine sense codons, one stop codon is converted to a synonymous stop codon, and the cognate tRNA-encoding genes and RF-encoding genes are removed.

FIG. 5—Depicts a flow diagram for training and deploying a machine learning model for designing a recoded organism

FIG. 6—Depicts example training data used to train a machine learning model.

FIG. 7—Illustrates an example computing device 300 for implementing the methods described above in relation to FIGS. 5 and 6.

### DETAILED DESCRIPTION OF THE INVENTION

A sequence listing forms part of the disclosure of this application and is incorporated as part of the disclosure.

The inventors have developed methods to produce biomanufactured products such as nucleotides, amino acids, their polymers, small molecules, metabolites and other molecules in engineered organisms such as recoiled organisms that are optimized for release into open environments, as defined herein. These organisms can be derived from bacteria such as E. coli.

Biomanufactured Products (BPs)

“Biomanufactured products” or “BPs” are products that are biomanufactured in entities. In some embodiments, a single product consists of many parts to be manufactured in more than one entity and combined downstream. In some embodiments, a single product consists of many parts to be manufactured in a single entity and combined within the entity. In some embodiments, a single product consists of only one part. The BPs that can be made according to the invention are unlimited in purpose.

Preferably, the BP biomanufactured by the method disclosed herein is derived directly or indirectly from an exogenous nucleic acid that is introduced into the cell. The term “exogenous” refers to anything that is introduced into an organism or a cell. An “exogenous nucleic acid” is a nucleic acid that entered a bacterium or other organism, or cell type, through the cell wall or cell membrane. An exogenous nucleic acid may contain a nucleotide sequence that exists in the native genome of an organism or a cell and/or nucleotide sequences that did not previously exist in the organism's or cell's genome. Exogenous nucleic acids include exogenous genes. An “exogenous gene” is a nucleic acid that codes for the expression of an RNA and/or protein that has been introduced into an organism or a cell (e.g., by transformation/transfection), and is also referred to as a “transgene.”

Nucleotides and Nucleic Acids

As is known in the art, modifications to nucleic acids (e.g., DNA and RNA) are provided that are not detrimental to their use and function. Thus, useful nucleic acids according to the present invention may have the sequences which are shown in the sequence listing or they may be slightly different. For example, useful nucleic acids may be at least 99 percent, at least 98 percent, at least 97 percent, at least 96 percent, at least 95 percent, at least 94 percent, at least 93 percent, at least 92 percent, at least 91 percent, at least 90 percent, at least 89 percent, at least 88 percent, at least 87 percent, at least 86 percent, at least 85 percent, at least 84 percent, at least 83 percent, at least 82 percent, 81 percent, or at least 80 percent identical. Generally, the length of the nucleic acid of the present invention is greater than about 30 nucleotides in length (e.g., at least or greater than about 35, 40, 45, 50, 55, 60, 70, 80, 90, 100, 120, 140, 160, 180, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, and 3,000, 4,000, 5,000, 6,000, 7,000, 8,000, 9,000, 10,000, 20,000, 30,000, 40,000, 50,000, 60,000, 70,000, 80,000, 90,000 or up to and including 100,000 nucleotides)

In certain embodiments, the BP biomanufactured by the method disclosed herein comprises a nucleic acid (e.g., DNA or RNA). Examples of nucleotides or nucleic acids include NTPs, dNTPs, plasmids, nanoplasmids, linearized vectors, minicircles, bacmid DNA, mRNA, and circRNA.

The term “plasmid” refers to a circular DNA molecule that is physically separate from an organism's genomic DNA. Plasmids may be linearized before being introduced into a host cell (referred to herein as a linearized plasmid). Linearized plasmids may not be self-replicating, but may integrate into and be replicated with the genomic DNA of an organism. The term “vector,” as used herein, is intended to refer to a nucleic acid molecule capable of transporting another nucleic acid to which it has been linked. One type of vector is a “plasmid”, which refers to a circular double stranded DNA loop into which additional DNA segments may be ligated. Another type of vector is a phage vector. Another type of vector is a viral vector, wherein additional DNA segments may be ligated into the viral genome. A vector is capable of transferring nucleic acid sequences to target cells. For example, a vector may comprise a coding sequence capable of being expressed in a target cell. For the purposes of the present invention, “vector construct,” “expression vector,” and “gene transfer vector,” generally refer to any nucleic acid construct capable of directing the expression of a gene of interest and which is useful in transferring the gene of interest into target cells. Thus, the term includes cloning and expression vehicles, as well as integrating vectors. A “minicircle” vector, as used herein, refers to a small, double stranded circular DNA molecule that provides for persistent, high level expression of a sequence of interest that is present on the vector, which sequence of interest may encode a polypeptide, an shRNA, an anti-sense RNA, an siRNA, and the like in a manner that is at least substantially expression cassette sequence and direction independent. The sequence of interest is operably linked to regulatory sequences present on the mini-circle vector, which regulatory sequences control its expression. Such mini-circle vectors are described, for example, in published U.S. Patent Application US20040214329, herein specifically incorporated by reference.

Amino Acids and their Polymers

As is further known in the art, modifications to amino acid polymers including allelic variations and polymorphisms may occur in parts of proteins that are not detrimental to their use and function. Thus, useful amino acid polymers according to the present invention may have the sequences which are shown in the sequence listing or they may be slightly different. For example, useful amino acid polymers may be at least 99 percent, at least 98 percent, at least 97 percent, at least 96 percent, at least 95 percent, at least 94 percent, at least 93 percent, at least 92 percent, at least 91 percent, at least 90 percent, at least 89 percent, at least 88 percent, at least 87 percent, at least 86 percent, at least 85 percent, at least 84 percent, at least 83 percent, at least 82 percent, 81 percent, or at least 80 percent identical.

In certain embodiments, the BP produced by the method disclosed herein comprises a polypeptide or protein. Examples of amino acids or their polymers include antigenic polypeptides or proteins (e.g., viral protein components as vaccines), antibodies, nanobodies, enzymatic proteins, cytokines, endocrine proteins, signaling proteins, scaffolding proteins, etc.

In certain embodiments, the BP produced by the method disclosed herein comprises a biologic polypeptide or protein. As used herein, a “biologic” is a polypeptide-based molecule produced by the methods provided herein and which may be used to treat, cure, mitigate, prevent, or diagnose a serious or life-threatening disease or medical condition. Biologics, according to the present invention include, but are not limited to, allergenic extracts, blood components, gene therapy products, human tissue or cellular products used in transplantation, vaccines, antibodies, cytokines, growth factors, enzymes, thrombolytics, and immunomodulators, among others. A biologic polypeptide of the present invention may be utilized to treat conditions or diseases in many therapeutic areas such as, but not limited to, blood, cardiovascular, CNS, dermatology, endocrinology, genetic, genitourinary, gastrointestinal, musculoskeletal, oncology, and immunology, respiratory, sensory and anti-infectives.

The term “human antibody”, as used herein, is intended to include antibodies having variable regions in which both the framework and CDR regions are derived from sequences of human origin. Furthermore, if the antibody contains a constant region, the constant region also is derived from such human sequences, e.g. human germline sequences, or mutated versions of human germline sequences or antibody containing consensus framework sequences derived from human framework sequences analysis, for example, as previously described¹. The term “recombinant human antibody”, as used herein, includes all human antibodies that are prepared, expressed, created or isolated by recombinant means, such as antibodies isolated from an animal (e.g. a mouse) that is transgenic or transchromosomal for human immunoglobulin genes or a hybridoma prepared therefrom, antibodies isolated from a host cell transformed to express the human antibody, antibodies isolated from a recombinant, combinatorial human antibody library, and antibodies prepared, expressed, created or isolated by any other means that involve splicing of all or a portion of a human immunoglobulin gene. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

Examples of cytokines and growth factors of interest include, but are not limited to, insulin, insulin-like growth factor, hGH, tPA, interleukins (IL), e.g., IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, interferon (IFN) alpha, IFN beta, IFN gamma, IFN omega or IFN tau, tumor necrosis factor (TNF), such as TNF alpha and TNF beta, TNF gamma, TRAIL, G-CSF, GM-CSF, M-CSF, MCP-1 and VEGF.

Antigenic polypeptides include any polypeptide from a human pathogen. In certain embodiments, the pathogen is a viral pathogen, a bacterial pathogen, a fungal pathogen, a parasitic helminth, or a parasitic protozoan. In some embodiments, the viral pathogen is wild-type or recombinant virus, of any type of strain, chosen from the orthomyxoviridae virus family, including in particular flu viruses, such as mammalian influenza viruses, and more particularly human influenza viruses, porcine influenza viruses, equine influenza viruses, feline influenza viruses, avian influenza viruses, such as the swan influenza virus, the paramyxoviridae virus family, including respiroviruses (sendai, bovine parainfluenza virus 3, human parainfluenza 1 and 3), rubulaviruses (human parainfluenza 2, 4, 4a, 4b, the human mumps virus, parainfluenza type 5), avulaviruses (Newcastle disease virus (NDV)), pneumoviruses (human and bovine respiratory syncytial viruses), metapneumoviruses (animal and human metapneumoviruses), morbiliviruses (measle virus, distemper virus and rinderpest virus) and henipaviruses (Hendra virus, nipah virus, etc.), the coronaviridae virus family including in particular human coronaviruses (in particular NL63, SARS-CoV, MERS-CoV) and animal coronaviruses (canine, porcine, bovine coronaviruses and avian infectious bronchitis coronavirus), the flaviviridae virus family including in particular arboviruses (tick-borne encephalitis virus), flaviviruses (dengue virus, yellow fever virus, Saint Louis encephalitis virus, Japanese encephalitis virus, West Nile virus including the Kunjin subtype, Muray valley virus, Rocio virus, Ilheus virus, tick-borne meningo-encephalitis virus), hepaciviruses (hepatitis C virus, hepatitis A virus, hepatitis B virus) and pestiviruses (border disease virus, bovine diarrhea virus, swan fever virus), the Rhabdoviridae viruses including in particular vesiculoviruses (vesicular stomatitis virus), lyssaviruses (Australian, European Lagos bat virus, rabies virus), ephemeroviruses (bovine ephemeral fever virus), novirhabdoviruses (snakehead virus, hemorrhagic septicemia virus and hematopoietic necrosis virus), the Togaviridae virus family including in particular rubiviruses (rubella virus), alphaviruses (in particular Sinbis virus, Semliki forest virus, O'nyong'nyong virus, Chikungunya virus, Mayaro virus, Ross river virus, Eastern equine encephalitis virus, Western equine encephalitis virus, Venezuela equine encephalitis virus), the herpesviridae virus family including in particular human herpesviruses (HSV-1, HSV-2, chicken pox virus, Epstein-Barr virus, cytomegalovirus, roseolovirus, HHV-7 and KSHV), the poxviridae virus family including in particular orthopoxviruses (such as in particular camoepox, cowpox, smallpox, vaccinia), carpipoxviruses (including in particular sheep pox), avipoxviruses (including in particular fowlpox), parapoxviruses (including in particular bovine papular stomatitis virus) and leporipoxviruses (including in particular myxomatosis virus), the retroviridae virus family including in particular lentiviruses (including in particular human, feline and simian immunodeficiency viruses 1 and 2, caprine arthritis encephalitis virus or Maedi-Visna disease virus) and retroviruses (including in particular Rous sarcoma virus, human lymphotrophic viruses 1, 2 and 3). In some embodiments, the bacterial pathogen is Helicobacter pylori, Borrelia burgdorferi (Lyme disease), Escherichia coli, Mycobacteria tuberculosis, Staphylococcus aureus, Neisseria gonorrhoeae, Streptococcus pneumoniae, Corynebacterium diphtheria, or Vibrio cholera. In some embodiments, the fungal pathogen is Candida albicans. In some embodiments, the protozoan parasite is Plasmodium falciparum, Trypanosoma cruzi, Giardia lamblia, Toxoplasma gondii, Trichomonas vaginalis, or Entamoeba histolytica. In some embodiments, the helminth is Strongyloides stercoralis, Onchocerca volvulus, Loa loa, or Wuchereria bancrofti.

Also provided are auto-antigen polypeptides associated with any one of a number of autoimmune diseases, such as but not limited to, Sjogren's syndrome, type 1 diabetes, rheumatoid arthritis, systemic lupus erythematosus, celiac disease, myasthenia gravis, Hashimoto's thyroiditis, Graves' disease, autoimmune polyendocrinopathy-candidiasis-ectodermal dystrophy (APECED), disseminated non-tuberculosis mycobacterial (dNTM) infection, or any other autoimmune disease including 21-hydroxylase deficiency, acute anterior uveitis, acute disseminated encephalomyelitis (ADEM), acute necrotizing hemorrhagic leukoencephalitis, Addison's disease, gammaglobulinemia, alopecia areata, amyloidosis, ankylosing spondylitis, anti-GBM/Anti-TBM nephritis, antiphospholipid syndrome (APS), autoimmune angioedema, autoimmune aplastic anemia, autoimmune dysautonomia, autoimmune hepatitis, autoimmune hyperlipidemia, autoimmune immunodeficiency, autoimmune inner ear disease (AIED), autoimmune myocarditis, autoimmune oophoritis, autoimmune pancreatitis, autoimmune retinopathy, autoimmune thrombocytopenic purpura (ATP), autoimmune thyroid disease, autoimmune urticarial, axonal and neuronal neuropathies, Balo disease, Behcet's disease, bullous pemphigoid, cardiomyopathy, Castleman disease, celiac disease, Chagas disease, chronic inflammatory demyelinating polyneuropathy (CIDP), chronic recurrent multifocal ostomyelitis (CRMO), Churg-Strauss syndrome, cicatricial pemphigoid/benign mucosal pemphigoid, Crohn's disease, Cogans syndrome, cold agglutinin disease, congenital heart block, coxsackie myocarditis, CREST disease, cryoglobulinemia, demyelinating neuropathies, dermatitis herpetiformis, dermatomyositis, Devic's disease (neuromyelitis optica), discoid lupus, Dressler's syndrome, endometriosis, eosinophilic esophagitis, eosinophilic fasciitis, erythema nodosum, experimental allergic encephalomyelitis, Evans syndrome, fibrosing alveolitis, giant cell arteritis (temporal arteritis), giant cell myocarditis, glomerulonephritis, Goodpasture's syndrome, granulomatosis with polyangiitis (GPA), Graves' disease, Guillain-Barre syndrome, Hashimoto's encephalitis, Hashimoto's thyroiditis, hemolytic anemia, Henoch-Schonlein purpura, herpes gestationis, hypogammaglobulinemia, idiopathic thrombocytopenic purpura (ITP), IgA nephropathy, IgG4-related sclerosing disease, immunoregulatory lipoproteins, inclusion body myositis, inflammatory bowel disease, interstitial cystitis, juvenile arthritis, juvenile diabetes (type 1 diabetes), juvenile myositis, Kawasaki syndrome, Lambert-Eaton syndrome, leukocytoclastic vasculitis, lichen planus, lichen sclerosus, ligneous conjunctivitis, linear IgA disease (LAD), membranous nephropathy, Meniere's disease, microscopic polyangiitis, mixed connective tissue disease (MCTD), Mooren's ulcer, Mucha-Habermann disease, multiple sclerosis, myasthenia gravis, myositis, narcolepsy, neutropenia, ocular cicatricial pemphigoid, optic neuritis, palindromic rheumatism, pediatric autoimmune neuropsychiatric disorders associated with streptococcus (PANDAS), paraneoplastic cerebellar degeneration, paroxysmal nocturnal hemoglobinuria (PNH), Parry Romberg syndrome, Parsonnage-Turner syndrome, pars planitis (peripheral uveitis), pemphigus, peripheral neuropathy, perivenous encephalomyelitis, pernicious anemia, POEMS syndrome, polyarteritis nodosa, type I, II, & III autoimmune polyglandular syndromes, polymyalgia rheumatic, polymyositis, postmyocardial infarction syndrome, postpericardiotomy syndrome, progesterone dermatitis, primary biliary cirrhosis, primary sclerosing cholangitis, psoriasis, psoriatic arthritis, pulmonary fibrosis (idiopathic), pyoderma gangrenosum, pure red cell aplasia, Raynaud's phenomenon, reactive arthritis, reflex sympathetic dystrophy, Reiter's syndrome, relapsing polychondritis, restless legs syndrome, retroperitoneal fibrosis, rheumatic fever, rheumatoid arthritis, sarcoidosis, Schmidt syndrome, scleritis, scleroderma, Sjogren's syndrome, sperm and testicular autoimmunity, stiff person syndrome, subacute bacterial endocarditis (SBE), Susac's syndrome, sympathetic ophthalmia, systemic lupus erythematosus (SLE), Takayasu's arteritis, temporal arteritis/Giant cell arteritis, thrombocytopenic purpura (TTP), Tolosa-Hunt syndrome, transverse myelitis, type 1 diabetes, ulcerative colitis, undifferentiated connective tissue disease (UCTD), uveitis, vasculitis, vesiculobullous dermatosis, and vitiligo.

Also provided are nutritional or nutritive compositions. A composition, formulation or product is “nutritional” or “nutritive” if it provides an appreciable amount of nourishment to its intended consumer, meaning the consumer assimilates all or a portion of the composition or formulation into a cell, organ, and/or tissue. Generally, such assimilation into a cell, organ and/or tissue provides a benefit or utility to the consumer, e.g., by maintaining or improving the health and/or natural function(s) of said cell, organ, and/or tissue. A nutritional composition or formulation that is assimilated as described herein is termed “nutrition.” By way of non-limiting example, a polypeptide is nutritional if it provides an appreciable amount of polypeptide nourishment to its intended consumer, meaning the consumer assimilates all or a portion of the protein, typically in the form of single amino acids or small peptides, into a cell, organ, and/or tissue. “Nutrition” also means the process of providing to a subject, such as a human or other mammal, a nutritional composition, formulation, product or other material. A nutritional product need not be “nutritionally complete,” meaning if consumed in sufficient quantity, the product provides all carbohydrates, lipids, essential fatty acids, essential amino acids, conditionally essential amino acids, vitamins, and minerals required for health of the consumer. Additionally, a “nutritionally complete protein” contains all protein nutrition required (meaning the amount required for physiological normalcy by the organism) but does not necessarily contain micronutrients such as vitamins and minerals, carbohydrates or lipids. For example, a nutritional benefit is the benefit to a consuming organism equivalent to or greater than at least about 0.5% of a reference daily intake value of protein, such as about 1%, 2%, 3%, 4%, 5%, 6%, 7%, 8%, 9%, 10%, 15%, 20%, 25%, 30%, 35%, 40%, 45%, 50%, 60%, 65%, 70%, 75%, 80%, 85%, 90%, 95%, 100% or greater than about 100% of a reference daily intake value.

In some embodiments the nutritive protein is an abundant protein in food. In some embodiments the abundant protein in food is selected from chicken egg proteins such as ovalbumin, ovotransferrin, and ovomucuoid; meat proteins such as myosin, actin, tropomyosin, collagen, and troponin; cereal proteins such as casein, alpha1 casein, alpha2 casein, beta casein, kappa casein, beta-lactoglobulin, alpha-lactalbumin, glycinin, beta-conglycinin, glutelin, prolamine, gliadin, glutenin, albumin, globulin; chicken muscle proteins such as albumin, enolase, creatine kinase, phosphoglycerate mutase, triosephosphate isomerase, apolipoprotein, ovotransferrin, phosphoglucomutase, phosphoglycerate kinase, glycerol-3-phosphate dehydrogenase, glyceraldehyde 3-phosphate dehydrogenase, hemoglobin, cofilin, glycogen phosphorylase, fructose-1,6-bisphosphatase, actin, myosin, tropomyosin a-chain, casein kinase, glycogen phosphorylase, fructose-1,6-bisphosphatase, aldolase, tubulin, vimentin, endoplasmin, lactate dehydrogenase, dextrin, transthyretin, fructose bisphosphate aldolase, carbonic anhydrase, aldehyde dehydrogenase, annexin, adenosyl homocvsteinase; pork muscle proteins such as actin, myosin, enolase, titin, cofilin, phosphoglycerate kinase, enolase, pyruvate dehydrogenase, glycogen phosphorylase, triosephosphate isomerase, myokinase; and fish proteins such as parvalbumin, pyruvate dehydrogenase, desmin, and triosephosphate isomerase.

In some aspects the nutritive polypeptide is selected to have a desired density of branched chain amino acids (BCAA). For example, BCAA density, either individual BCAAs or total BCAA content is about equal to or greater than the density of branched chain amino acids present in a full-length reference nutritional polypeptide, such as bovine lactoglobulin, bovine beta-casein or bovine type I collagen, e.g., BCAA density in a nutritive polypeptide is at least about 5%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500% or above 500% greater than a reference nutritional polypeptide or the polypeptide present in an agriculturally-derived food product. BCAA density in a nutritive polypeptide can also be selected for in combination with one or more attributes such as EAA density.

In some aspects the nutritive polypeptide is selected to have a desired density of one or more essential amino acids (EAA). Essential amino acid deficiency can be treated or, prevented with the effective administration of the one or more essential amino acids otherwise absent or present in insufficient amounts in a subject's diet. For example, EAA density is about equal to or greater than the density of essential amino acids present in a full-length reference nutritional polypeptide, such as bovine lactoglobulin, bovine beta-casein or bovine type I collagen, e.g., EAA density in a nutritive polypeptide is at least about 5%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500% or above 500% greater than a reference nutritional polypeptide or the polypeptide present in an agriculturally-derived food product.

In some aspects the nutritive polypeptide is selected to have a desired density of aromatic amino acids (“AAA”, including phenylalanine, tryptophan, tyrosine, histidine, and thyroxine). AAAs are useful, e.g., in neurological development and prevention of exercise-induced fatigue. For example, AAA density is about equal to or greater than the density of essential amino acids present in a full-length reference nutritional polypeptide, such as bovine lactoglobulin, bovine beta-casein or bovine type I collagen, e.g., AAA density in a nutritive polypeptide is at least about 5%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500% or above 500% greater than a reference nutritional polypeptide or the polypeptide present in an agriculturally-derived food product.

In some embodiments a protein comprises or consists of a derivative or mutein of a protein or fragment of an edible species protein or a protein that naturally occurs in a food product.

Such a protein can be referred to as an “engineered protein.” In such embodiments the natural protein or fragment thereof is a “reference” protein or polypeptide and the engineered protein or a first polypeptide sequence thereof comprises at least one sequence modification relative to the amino acid sequence of the reference protein or polypeptide. For example, in some embodiments the engineered protein or first polypeptide sequence thereof is at least 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, 99%, or 99.5% identical to at least one reference protein amino acid sequence. Typically the ratio of at least one of branched chain amino acid residues to total amino acid residues, essential amino acid residues to total amino acid residues, and leucine residues to total amino acid residues, present in the engineered protein or a first polypeptide sequence thereof is greater than the corresponding ratio of at least one of branched chain amino acid residues to total amino acid residues, essential amino acid residues to total amino acid residues, and leucine residues to total amino acid residues present in the reference protein or polypeptide sequence.

Industrial enzymes include oxidoreductases (e.g., dehydrogenases, oxidases, oxygenases, peroxidases), transferases (e.g., fructosyltransferases, transketolases, acyltransferases, transaminases), hydrolases (e.g., proteases, amylases, acylases, lipases, phosphatases, cutinases), lyases (pectate lyases, hydratases, dehydratases, decarboxylases, fumarase, arginosuccinases), isomerases (isomerases, epimerases, racemases), and ligases (e.g., synthetases, ligases).

Small Molecules and Metabolites

In certain embodiments, the BP biomanufactured by the method disclosed herein comprises a small molecule or metabolite. In certain embodiments, the BP biomanufactured by the method disclosed herein comprises a small molecule or metabolite.

Small molecules and metabolites can be any that are known to skill in the art. They can include but are not limited to amino acids, dNTPs, NTPs, and vitamins.

Metabolic reactions utilize the activity of cytochrome P450 monooxygenases² (CYPs) and uridine diphosphoglucuronosyltransferases (UGTs) as well as dehydrogenases, hydrolases, glutathione transferases, sulfotransferases, flavin monooxygenases, aldehyde oxidase, xanthine oxidoreductase, and others.

Entities, Engineered Organisms (EOs), Biomanufacturing Engineered Organisms (REOs), Genome Designs, and Functional Properties

As used herein, the term “engineered organism” or “EO” refers to an organism engineered from an original organism or “entity” to change or impart a “functional property” (e.g., to acquire a useful function or functions). It is understood that an EO may have a plurality of functional properties compared to a corresponding entity. In one embodiment, the entity from which the EO is engineered, is a wild type organism (“wild type entity”). In another embodiment, the entity from which the EO is engineered has already been engineered previously such that it contains existing introduced mutations (“engineered entity”). In another embodiment, the entity from which the EO is engineered has already been engineered previously such that it contains existing introduced mutations and is itself an EO. In some embodiments, the entity is a base strain.

As used herein, the term “released engineered organism” or “REO” refers to an organism that is fully proficient for biomanufacturing of a BP. It is understood that the REO is generated by engineering an EO. It is understood that the entity that the customer currently uses for biomanufacturing of a BP is also fully proficient for biomanufacturing of the BP and is referred to herein a “base strain”. It is understood that use of an REO is not limited to a biomanufacturing context. Rather, an REO can be used to biomanufacture a BP without isolating or purifying the BP, for example, in an open environment. In this context, culturing art REO is also useful for amplifying an REO population, for example, to generate large amounts of the REO prior to using it in an open environment. As described herein, this process is referred to as “culturing” the REO, for clarity. REOs are suitable for culturing using current good manufacturing practices (cGMP) or non-cGMP conditions. In certain embodiments, the REO comprises at least one additional or modified nucleic acid sequence or element relative to the EO, that encodes the at least one BP to be biomanufactured in the REO.

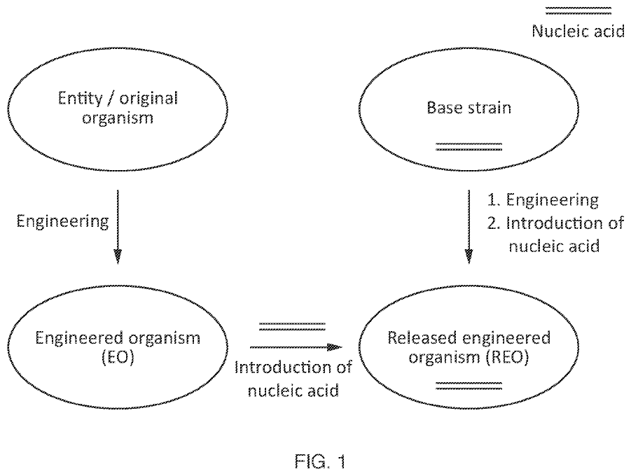

Other than the at least one additional or modified nucleic acid sequence or element in the REO that encodes the at least one BP to be biomanufactured in the REO, the REO optionally may contain at least one additional or modified nucleic acid sequence or element relative to the EO, such that the: 1) REO generally looks and behaves more similarly to the specific base strain than the EO does, or such that the 2) REO's target functional property remains equivalent or enhanced relative to the EO. In some embodiments, the REO contains both types of optional modifications. In some embodiments, the REO contains a plurality of these modifications. It is understood that if the modifications described in 1) and 2) are present in the REO, that in some embodiments, these modifications can be defined as part of the genetic material comprising the EO as well. The relationship between entities, base strains, EOs and REOs, is illustrated in FIG. 1.

Entities, EOs, and REOs can be of any genus, species or strain that can be engineered. In certain embodiments, the entity, EO or BEO is a prokaryote (e.g., a bacterium), including but not limited to: Escherichia coli, Escherichia coli NGF-1, Escherichia coli UU2685, Escherichia coli K-12 MG1655, Escherichia coli “recoded” or “GRO” strains and derivatives³⁻¹⁴, Escherichia coli C7 strains^{6, 7}, Escherichia coli C7ΔA strains⁵⁻⁷, Escherichia coli C13 strains^{5, 6}, Escherichia coli C13ΔA strains^{5, 6}, Escherichia coli “C321 strains”^{5, 6, 8-11}, Escherichia coli C321ΔA strains^{5, 6, 8-11}, Escherichia coli C321ΔA “synthetic auxotroph” strains and derivatives^{10, 11}, Escherichia coli evolved C321 strains^{8, 9}, Escherichia coli C321.ΔA.M9adapted strains⁸, Escherichia coli C321.ΔA.opt strains⁹, Escherichia coli rE.coli-57 strains and derivatives³, Escherichia coli C321ΔA “Syn61” strains and derivatives¹³, Escherichia coli K-12 MG1655 “MDS” strains and derivatives¹⁵⁻¹⁷, Escherichia coli K-12 MG1655 MDS9 strains¹⁵⁻¹⁷, Escherichia coli K-12 MG1655 MDS12 strains¹⁵⁻¹⁷, Escherichia coli K-12 MG1655 MDS41 strains¹⁵⁻¹⁷, Escherichia coli K-12 MG1655 MDS42 strains¹⁵⁻¹⁷, Escherichia coli K-12 MG1655 MDS43 strains¹⁵⁻¹⁷, Escherichia coli K-12 MG1655 MDS66 strains¹⁵⁻¹⁷, Escherichia coli BL21 DE3, Escherichia coli BL21 hybrid strains (“BLK strains”)¹⁵⁻¹⁷, Escherichia coli Nissle 1917, Salmonella, Salmonella typhimurium, Salmonella typhi Ty21a, Lactobacillus, Lactobacillus plantarum, Lactobacillus reuteri, Lactobacillus gasseri, Lactobacillus gasseri BNR17, Lactobacillus fermentum KLD, Lactobacillus helveticus, Lactobacillus helveticus strain NS8, Lactococcus, Lactococcus lactis, Lactococcus lactis NZ9000, Lactococcus NZ3900, Lactococcus lactis NZ9001, Lactococcus lactis MG1363, Bacteroides, Bacteroides thetaiotaomicron, Bacteroides fragilis, Bacteroides vulgatus, Bacteroides ovatus, Bacteroides uniformis, Bacteroides eggerthii, Bacteroides xylanisolvens, Bacteroides intestinalis, Bacteroides dorei, Bacteroides cellulosilyticus, Bacillus, Bacillus subtilis, Acetobacter, Streptomyces, Streptococcus, Staphylococcus, Staphylococcus epidermis, Bifidobacterium, Bifidobacterium longum, Bifidobacterium infantis, Eubacterium, Corynebacterium, Corynebacterium glutamicum, Rumunococcus, Coprococcus, Fusobacterium, Clostridium, Clostridium butyricum, Shewanella, Cyanobacterium, Mycoplasma, Mycoplasma capricolum, Mycoplasma genitalium, Mycoplasma mycoides, Mycoplasma mycoides JCVI-syn strains^{18, 19}, Mycoplasma mycoides JCVI-syn3.0 strains¹⁹, Listeria, Listeria monocytogenes, Vibrio, Vibrio cholerae, Vibrio natriegens, Vibrio natriegens Vmax strains²⁰, Pseudomonas. It is understood that any strains that are derivatives of or that are evolved from the strains in this listing, are also included in this listing for the purpose of this invention. Notably, a modified strain whose genome is at least 20%, 25%, 30%, 35%, 40%, 45%, 50%, 55%, 60%, 65%, 70%, 75%, 80%, 85%, 90%, 95%, 96%, 97%, 98%, 99%, 99.9%, or 99.99% identical to the genomic sequence of an aforementioned strain is understood to be of the same strain. References are included for different strains for the purpose of example only, and are not meant to limit the strain listing in any way. It is understood that higher organisms, such as yeast and mammalian cells can also be used.

In certain embodiments, the entity, EO or REO comprises genetic material present within the genome. In certain embodiments, the entity, EO or REO comprises genetic material that is non-genomic or episomal. In certain embodiments, a plurality of types of genetic material are present.

As used herein, an element is used to define a nucleic acid sequence by the functional product resulting from it. For example, an element can include a nucleic acid sequence that is described by its resulting polypeptide or other final functional unit such as a transposable element. It is understood that “native” means it occurs generally in nature, and “synthetic” means it does not occur generally in nature. In certain embodiments, the genetic material comprises at least one “native” nucleic acid sequence or element. In certain embodiments, the genetic material comprises at least one “synthetic” nucleic acid sequence or element. In certain embodiments, a plurality of types of genetic material are present.

It is understood that “heterologous” means it does not occur naturally with respect to the specific entity, EO or REO. It is understood that “naturally occurring” means it does occur naturally with respect to the specific entity, EO or REO. In certain embodiments, the genetic material comprises at least one heterologous nucleic acid sequence or element. In certain embodiments, the genetic material comprises at least one naturally occurring nucleic acid sequence or element. In certain embodiments, a plurality of types of genetic material are present.

It is understood that “engineered” means any type of modification that can be made to a nucleic acid sequence. In certain embodiments, the genetic material comprises at least one engineered nucleic acid sequence or element.

In certain embodiments, a plurality of combinations and types of genetic material as described above and herein, may be present in a single entity, EO or REO.

In certain embodiments, the entity, EO or REO comprises genetic material comprised of at least one or a portion of one “orthogonal translation system” or “OTS”. It is understood that an OTS comprises an aminoacyl tRNA synthetase and cognate tRNA. In certain embodiments, the entity, EO or REO comprises genetic material comprised of at least one “suppressor tRNA”. It is understood that the at least one suppressor tRNA may be engineered. In certain embodiments, both are present. In certain embodiments, the at least one cognate tRNA of the OTS is engineered to recognize a specific codon. In certain embodiments, the at least one suppressor tRNA is engineered to recognize a specific codon. In certain embodiments a plurality of modifications may be present across these different types of genetic material.

It is understood that a “nonstandard amino acid” or “NSAA” is an amino acid that is not included in the twenty standard amino acids but may occur generally in nature. In certain embodiments, the NSAA does not occur generally in nature and is entirely synthetic. In certain embodiments, the at least one OTS incorporates an NSAA. In certain embodiments, the at least one OTS incorporates a standard amino acid. In certain embodiments, a suppressor tRNA incorporates a standard amino acid. In certain embodiments, the suppressor tRNA incorporates an NSAA. In certain embodiments, a plurality of these scenarios are true.

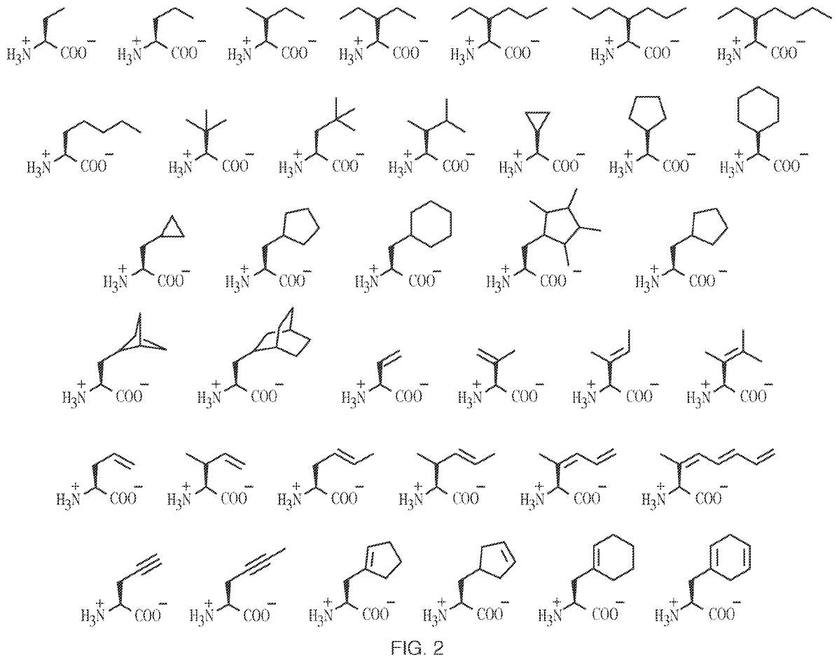

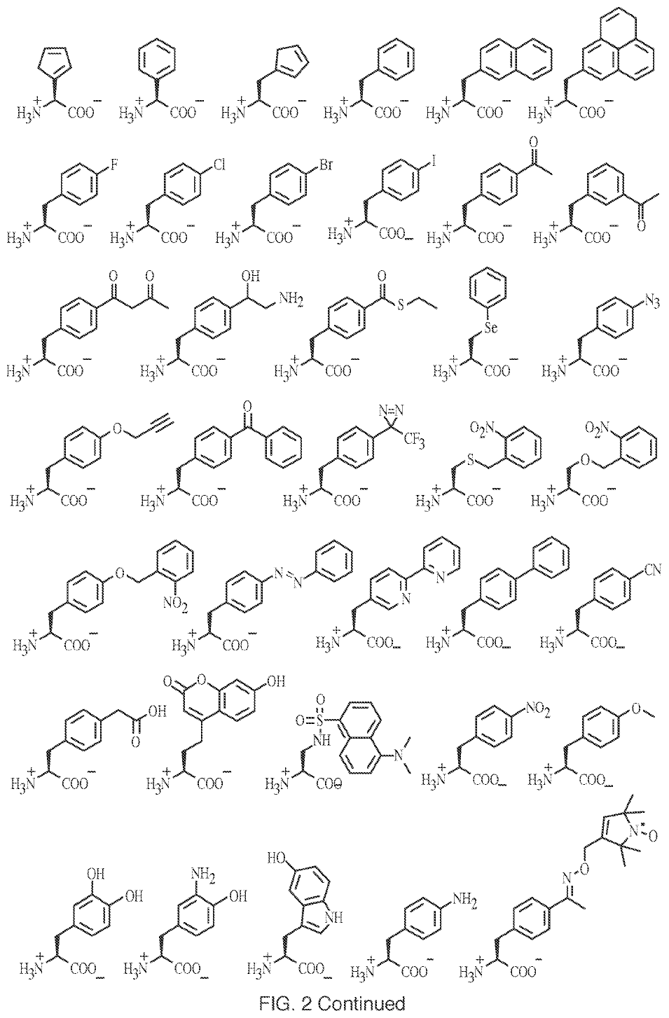

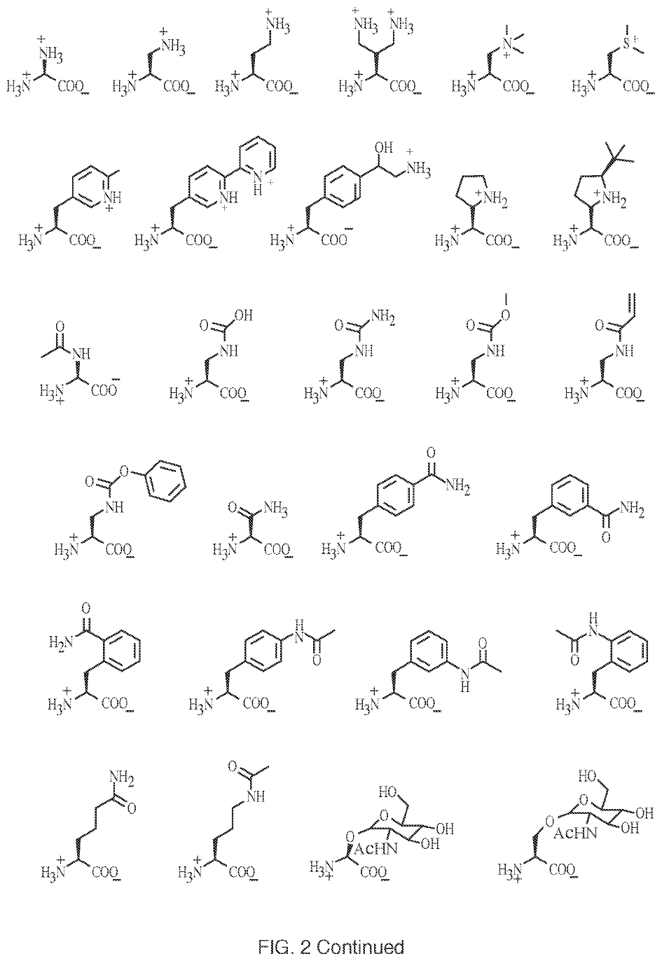

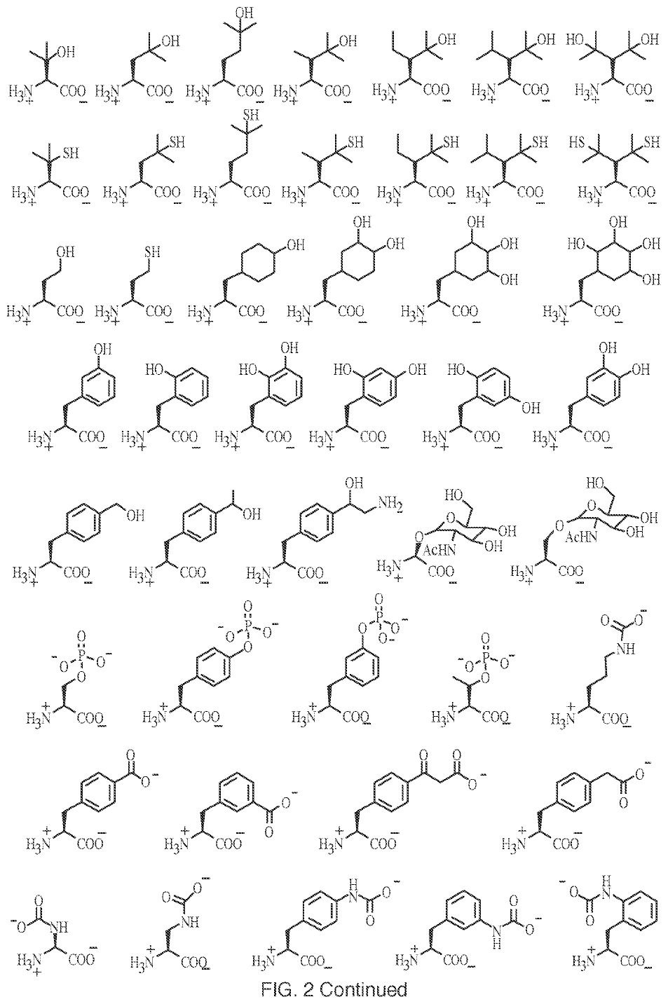

Exemplary NSAAs have been described²¹⁻²⁵ and a subset are listed herein in FIG. 2. Exemplary OTSs and suppressor tRNAs have also been described²⁶⁻²⁹. In certain embodiments, the NSAA is selected from the subset of the NSAA listed in FIG. 2 and those referenced herein.

The genetic material of EOs and REOs comprise both genomic and non-genomic material. It is understood that the genetic material comprising an EO can confer at least one functional property. It is understood that the genetic material comprising an EO can confer a plurality of functional properties. It is understood that the functional property of the EO can be conferred by a plurality of nucleic acid sequences comprising the genetic material. The at least one functional property can include but is not limited to one that makes the organism useful for biomanufacturing of at least one BP. It is understood that the at least one functional property of an EO may be generally desirable for biomanufacturing of various BPs. It is understood that the at least one functional property of an EO may be desirable for biomanufacturing of a specific BP. The “genome design” as described herein, is the specific sequence of nucleic acids that make up the genomic material of the EO. In some embodiments, the functional property conferred to the EO is specified by all or a portion of the genomic material. In some embodiments, the functional property conferred to the EO is specified by all or a portion of the non-genomic material. In some embodiments, the functional property conferred to the EO is specified by a plurality of combinations of genomic and non-genomic material. In some embodiments, the EO with the at least one functional property can be obtained via many different genome designs. In some embodiments, the EO with the at least one functional property can contain a genome design that comprises features from a plurality of different genome designs. It is also understood that the genome design of an entity can be engineered as part of the process of generating an EO.

It is understood that a plurality of genome designs and functional properties exist. Specific examples of genome designs as well as specific examples of functional properties, are described separately herein for the purpose of example only and not meant to limit the invention in any way. In some embodiments, for a given genome design, examples of functional properties imparted by it are listed for the purpose of example. In some embodiments, for a given functional property, examples of genome designs that can impart the functional property are listed for the purpose of example.

In certain embodiments, the REO is a probiotic organism, or probiotic. “Probiotic” is used to refer to live, non-pathogenic microorganisms, e.g., bacteria, which can confer health benefits to a host organism that contains an appropriate amount of the microorganism. In some embodiments, the host organism is a mammal. In some embodiments, the host organism is a human. Some species, strains, and/or subtypes of non-pathogenic bacteria are currently recognized as probiotic. Examples of probiotic bacteria include, but are not limited to, Bifidobacteria, Escherichia, Lactobacillus, and Saccharomyces. Some more specific examples include but are not limited to: Bifidobacterium bifidum, Enterococcus faecium, Escherichia coli, Lactobacillus acidophilus, Lactobacillus bulgaricus, Lactobacillus paracasei, Lactobacillus plantarum, and Saccharomyces boulardii. The probiotic may be a variant or a mutant strain of bacterium³⁰⁻³³. Non-pathogenic bacteria are engineered as provided herein to enhance or improve desired biological properties, for example, survivability. Non-pathogenic bacteria may be genetically engineered to provide probiotic properties. Probiotic bacteria may be engineered as provided herein to enhance or improve probiotic properties as described herein.

Genome Designs

Recoded Genome Designs

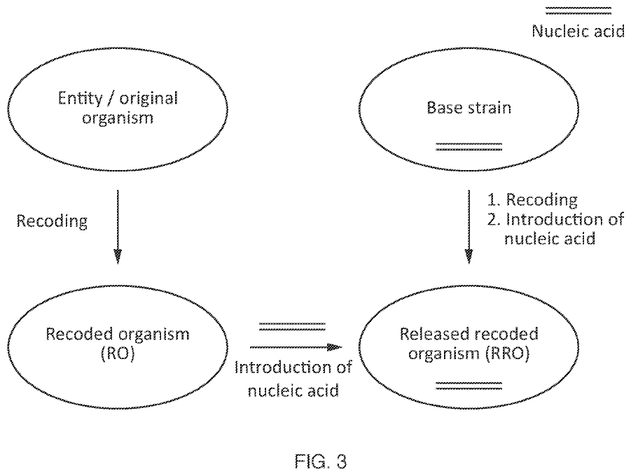

In certain embodiments, the genome design of the EO is a “recoded genome design”. In these embodiments, it is understood that the EO is a “recoded organism” or an “RO”, and that an RO is a type of EO. In these embodiments, it is also understood that the corresponding REO is a “released recoded organism” or “RRO”, and that a RRO is a type of REO. The relationship between entities, base strains, ROs and RROs, is illustrated in FIG. 3.

As used herein, the tern recoded organism or RO refers to an organism in which at least one “forbidden codon” has been partially or completely replaced with a “target synonymous codon” in the genome as previously described^{3, 5, 6, 13}. The forbidden and target synonymous codon can include a stop codon, sense codon or both types of codons. Complete replacement means replacement of all instances of the forbidden codon that occur throughout the genome. Partial replacement means replacement of any number of the forbidden codon less than all instances of the forbidden codon that occur throughout the genome. In certain embodiments, at least 0.0001%, 0.001%, 0.01%, 0.1%, 1%, 10%, 20%, 30%, 40%, 50%, 60%, 70%, 80%, 90%, 91%, 92%, 93%, 94%, 95%, 96%, 97%, 98%, or 99% of the forbidden codon in the genome is replaced by one or more synonymous codons. In certain embodiments, partial replacement means replacement of all forbidden codons that occur throughout essential genes. It is understood that in certain embodiments, “essential” means essential for viability. It is also understood that in certain embodiments, essential means essential for a reasonable level of fitness for the industrial application.

The RO can contain modifications of the forbidden codon directly within its genome or the genomic forbidden codons can be left untouched and the RO supplemented with non-genomic material such as one or many episomes that contain forbidden codons encoded as the target synonymous codon within their associated genes or genetic elements as described previously³⁴. In certain embodiments, the RO only contains modifications to forbidden codons within its genome. In certain embodiments, the RO only contains modifications using the episomal strategy. In certain embodiments, a combination of both strategies are used.

In certain embodiments, the RO further comprises a modification to at least one component of the translation machinery cognate to or corresponding to the replaced forbidden codon. It is understood that a modification can include deletion of the at least one component of the translation machinery. In certain embodiments where the replaced forbidden codon is a sense codon, the modified component of the translation machinery is a tRNA¹³ that recognizes the corresponding or cognate forbidden codon. In certain embodiments where the replaced forbidden codon is a stop codon, the modified component of the translation machinery is a release factor⁶ that recognizes the corresponding or cognate forbidden codon. In certain embodiments, one forbidden stop codon is completely replaced with the target synonymous codon and the corresponding or cognate release factor is deleted. In certain embodiments, one forbidden sense codon is completely replaced with the target synonymous codon and the corresponding or cognate tRNA is deleted. In certain embodiments, one forbidden stop codon is partially replaced with the target synonymous codon and the corresponding or cognate release factor is deleted. In certain embodiments, one forbidden sense codon is partially replaced with the target synonymous codon and the corresponding or cognate tRNA is deleted. In certain embodiments, one forbidden stop codon is completely replaced with the target synonymous codon and the corresponding or cognate release factor is deactivated or its specificity is modified such that its activity at the forbidden codon is lost. In certain embodiments, one forbidden sense codon is completely replaced with the target synonymous codon and the corresponding or cognate tRNA is deactivated or its specificity is modified such that its activity at the forbidden codon is lost. In certain embodiments, one forbidden stop codon is partially replaced with the target synonymous codon and the corresponding or cognate release factor is deactivated or its specificity is modified such that its activity at the forbidden codon is lost. In certain embodiments, one forbidden sense codon is partially replaced with the target synonymous codon and the corresponding or cognate tRNA is deactivated or its specificity is modified such that its activity at the forbidden codon is lost. In certain embodiments, a plurality of these scenarios mentioned are true in a single RO.

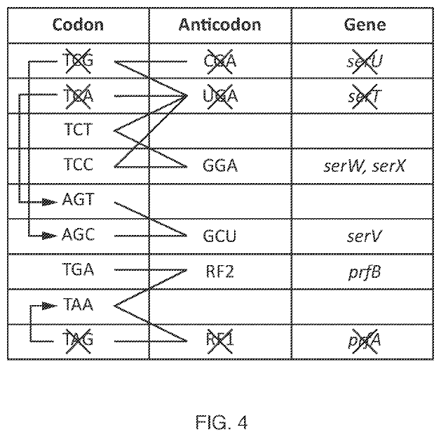

As an example, FIG. 4 illustrates a recoding scheme described previously¹³, whereby two serine sense codons are recoded to two synonymous serine sense codons, one stop codon is converted to a synonymous stop codon, and the cognate tRNA-encoding genes and RF-encoding genes are removed. This illustrates the means by which complete or partial replacement of a nonsense or sense codon to synonymous codons, can be completed to enable deletion of the cognate or corresponding components of the translation machinery without killing the cell. This methodology can be applied to many other sense codons or stop codons or a plurality of codons.

In certain embodiments, recoding designs can be “tightened” for various applications by additional modifications to the RO. In certain embodiments, the RO can be engineered to include a restriction enzyme within a restriction system, whereby the corresponding modification enzyme (typically a methylase) is absent and the restriction enzyme contains at least one forbidden codon. For example, the EcoRI restriction enzyme can be used for this purpose, whereby the host lacks the EcoRI methylase. If the RO lacks unwanted forbidden codon activity, the restriction enzyme is not active. If an event occurs in which unwanted forbidden codon activity arises, the associated forbidden codon in the restriction enzyme is expressed and any functional restriction enzyme produced kills the cell. This is a means by which cells containing the unwanted forbidden codon activity, potentially though some type of mutation event, for example, can be rid from the population. In certain embodiments, a similar mechanism can be used with toxin-antitoxin systems³⁵, where the antitoxin is absent and the toxin is only expressed during unwanted forbidden codon activity. In certain embodiments, multiple restriction systems can be modified in this way in a single RO. In certain embodiments, multiple toxin-antitoxin systems can be modified in this way in a single RO. In certain embodiments, a plurality of these modifications can be present within a single RO. Tightening of recoding designs can be useful for a variety of applications as described below. They can be used to protect a population against infection events by certain phages that harbor their own tRNAs³⁶. They can also be used as a general means to select against RO mutants in the population that contain mutations in translation machinery (e.g., unwanted tRNA suppressors that can read through forbidden codons or RF mutations that can expand specificity for forbidden stop codons) that would compromise the application for which the RO is used. Other embodiments can make similar use of, nucleases, proteases (and other degradative enzymes that are normally secreted but are toxic when expressed cytoplasmically without a signal sequence), restriction enzymes lacking their corresponding modification enzymes, phage proteins such as holins that are normally tightly repressed, and random peptides form libraries that are identified as toxic when expressed.

Notably, in certain cases as described herein, forbidden codon activity can be desired and also undesired in the same cell. A good example of this is with regard to phage resistance vs. codon encryption as described later. For example, tightened recoded designs can be used such that undesired codon activity by a phage at forbidden codon 1, kills the cell. In the same cell however, if forbidden codon 1 is also the site at which the codon is “encrypted” to produce a functional and desired product (e.g., transgene), forbidden codon meaning will conflict and the system will not work. In these such cases, a number of precautions can be taken: 1) This situation can be avoided by using ROs with many different forbidden codons, some that are used for the purpose of phage resistance and some that are used for codon encryption. In these embodiments, the forbidden codons used for phage resistance would not be reassigned or would keep their original (“old”) meaning, and the forbidden codons used for codon encryption would be reassigned with new meaning for the application. 2) Careful consideration can also be made with regard to the sites chosen for insertion of forbidden codons and the types of amino acids that are inserted. For example, if amino acid 1 is incorporated by a forbidden codon in a restriction enzyme and amino acid 2 is incorporated by the same forbidden codon in a transgene, the restriction enzyme should only function with insertion of amino acid 1 and not 2, and vice versa for the transgene.

Other Genome Designs

A large number of additional genome designs exist that can add, enhance, or modify EO functional properties. Examples of such genome designs are described in the “Functional Properties” section alongside associated functional properties that they confer. These genome designs are purely for the purpose of example and not meant to limit the invention in any way. Furthermore, although a given genome design may be described under a specific functional property, these genome designs impart many other functional properties in other sections or that are not described. A genome design's association with the listed functional property is meant for example only. In certain embodiments, a plurality of these genome designs, or “features” that are not defined as genome designs specifically, can be combined into a single genome design in an EO. In certain embodiments, a plurality of these genome designs can be combined into a single genome design in an EO that also incorporates a recoded genome design. Notably, depending on the desired functional property or plurality of functional properties, different genome designs or features thereof, will be appropriate.

Functional Properties

It is understood that the at least one functional property of an EO may be generally desirable for biomanufacturing of various BPs and for release into open environments. Such functional properties include but are not limited to: 1) inbound horizontal gene transfer blockage, 2) outbound horizontal gene transfer blockage, 3) biocontainment, and 4) NSAA incorporation.

Inbound and Outbound HGT Blockage

Inbound horizontal gene transfer (HGT) is a process by which any nucleic acid is transferred into a cell, such as an engineered cell or EO. Inbound HGT may occur by processes including but not limited to 1) transformation, whereby a cell takes up naked nucleic acid from the external environment, 2) phage infection, 3) phage transduction, in which non-phage DNA is packaged into a phage particle and injected into the cell of interest, 4) or by conjugation, in which another host cell transfers a portion of its DNA into the cell of interest. Thus, as defined herein, inbound HGT can include phage infection as well as transfer of non-phage nucleic acid, and typically involves transfer of DNA but may also apply to RNA, such as infection by an RNA virus.

Outbound HGT is any process by which the nucleic acid of a cell of interest is transferred to a second cell. Outbound HGT may occur by processes including but not limited to 1) transformation, whereby the cell of interest lyses and releases its nucleic acids, which are then taken up via the external environment into a second host, 2) phage transduction, in which non-phage DNA from the cell of interest is packaged into a phage particle and injected into another cell, or by 3) conjugation, in which the cell of interest transfers a portion of its DNA into another cell.

Unwanted Inbound HGT

Infection of EOs, REOs, or entities by “bacteriophages” or “phages” (viruses that infect bacteria) can occur during a culturing process and these infection events themselves can be extremely problematic. This can be significantly costly in terms of lost product, lost time, and lost money in the form of cost associated with cleaning the facility after the infection event, and lost revenue during the down time associated with facility cleaning. Each infection event is relatively more costly and problematic, from a regulatory perspective, if the REO is cultured with cGMP as opposed to research grade.

Inbound HGT can be problematic for other reasons as well. For example, phage transduction, that also occurs through phages, can bring unwanted genetic material from other EOs or REOs in the culturing facility into the target EO or REO that isn't meant to receive the genetic material. Phage-independent mechanisms can also mediate this transfer of information as described above. Either way, if this (often engineered) genetic material is shared with the REO, this could impact culturing processes in many ways. Biomanufacturing efficiencies could be impacted and unintended information sharing could have regulatory impacts as well.

Most of the existing approaches to blocking inbound HGT have focused on reducing phage infection events. If the phage can't infect a cell, the phage infection event itself will not impact the bioreactor, and any material it carries along with it (phage transduction), also can't be shared to an appreciable extent. Existing approaches to reducing phage infection events, have focused on the actual culturing process itself and also strain engineering improvements: 1) Preventative measures, for example those that involve extensive sterile technique, are often used that can slow down operations. The problem with this approach is that it decreases throughput, decreases revenue, and increases cost. 2) Phage receptor knock outs are also used to protect against infection by classes of phages that are known offenders of the facility. There are multiple problems with this approach. First, since different phages use different receptors, one receptor knock is unlikely to protect against all phages encountered in the facility. Second, some prior knowledge of the phages that are known to infect the facility is required for this approach to be successful. Third, phages evolve quickly to overcome these host mutations, resulting in a continuous battle whereby the strain is repeatedly modified to both counteract new phage infection events and existing ones. Fourth, phage receptor knock outs are also known to impair the fitness of strains, where fitness is important for many culturing processes and final application as a living therapeutic. Better mechanisms for reducing phage infection events are needed. Additionally, phages are only one mechanism by which inbound HGT can occur. Little has been done to address other mechanisms of inbound HGT as described herein and new approaches are needed to address this.

Unwanted Outbound HGT

Outbound HGT can play a role in the industrial culturing of REOs and is particularly concerning when the engineered genetic material contained within the EO or REO is shared with organisms in the open environment. As used herein, an “open environment” means any environment outside the culturing facility (“closed environment”). For REOs, there are two important open environments: 1) the environment just outside the culturing facility and 2) that in which the REO is used.

Outbound sharing of genetic material with organisms in the open environment just outside the culturing facility can occur through the unintended release of the EO or REO into that open environment. Time engineered genetic material within the EO or REO is then shared with other entities in that environment through non-phage-mediated or phage-mediated mechanisms as described herein. If the (often engineered) genetic material contained within the EO and REO is shared with organisms in the open environment, this engineered genetic material has the potential to cause unpredictable harm to the environment as well as entities therein. In some cases, depending on the environment, this could also be of concern to human health. For example, if the facility is located near a farm used to grow corn, or where cattle are being raised for beef consumption. Unintended release of EOs or REOs from the culturing facility, even at low levels, has the potential to be catastrophic to these open environments and since such low level release may be unavoidable in some cases, this deserves attention.

Outbound sharing of genetic material with native organisms or entities in the open environment in which the REO is used is highly problematic, especially if this environment is that of a human subject or an animal (e.g., the human gut). For example, the genetic material that is either directly or indirectly shared, could encode a BP that is only meant to be produced transiently in the gut by an RRO. In this case, the RRO may only be meant to exist transiently in the gut during a short therapeutic window. However, since this HGT event could unintentionally convert native organisms into “genetically modified organisms” or “GMOs” for sustained production of the BP, this could cause tremendous and ultimately unpredictable ham to the subject. Notably, this is only one example. For example, as living therapeutic markets grow, REOs are being increasingly deployed to treat a range of diseases from cancer to metabolic diseases. As these REOs are engineered with increasing complexity to address the growing need for new EOs with new functions, unregulated sharing of genetic material in this context is expected to represent a tremendous problem in the field and deserves attention. Further, there are many other examples of growing markets that involve REOs in open environments.

Outbound HGT can be problematic for other reasons as well. For example, phage transduction can carry unwanted genetic material out of the EO or REO in the culturing facility and into other EOs or REOs that weren't meant to receive the genetic material. Phage-independent mechanisms can also mediate this transfer of information as described above. Either way, if this (often engineered) genetic material is shared, this could impact culturing processes in many ways. Culturing efficiencies could be impacted and unintended information sharing could have regulatory impacts as well.

Most of the existing approaches to blocking outbound HGT have focused on reducing phage infection events. If the phage can't infect a cell, any material it carries along with it (phage transduction) also can't be shared to an appreciable extent. Existing approaches to reducing phage infection events, have focused on the actual culturing process itself and also strain engineering improvements as described above. As stated previously, better mechanisms for reducing phage infection events are needed. Additionally, phages are only one mechanism by which outbound HGT can occur. Little has been done to address other mechanisms of outbound HGT as described herein and new approaches are needed to address this.

Utility of Recoded Genome Designs

ROs naturally block some mechanisms of HGT and additional engineering to the RO can then be done to block other mechanisms of HGT.

Inbound HGT Blockage

Inbound HGT can occur through a number of mechanisms as described herein. One consequence of inbound HGT is the transfer of genetic material. This can occur through phages (transduction) and other mechanisms. Notably though, if the mechanism is via phage, the infection event itself can also be catastrophic. The use of recoded genome designs can be useful for generating EOs that are resistant to all forms of inbound HGT as described herein, and by extension, phage infection. ROs resist inbound HGT from any genetic material that contains forbidden codons, because such genetic material relies on translation machinery that has been modified or removed in the RO. As a result, the genetic material is not properly expressed. An example of this is described below as it relates to genetic material that is derived from a phage, but it is not meant to limit the invention in any way. By extension, similar embodiments can be drawn from this that involve other forms of genetic material (e.g., non-phage genetic material).

ROs can resist infection by phages whose genetic material contains forbidden codons because the phages rely on translation machinery that has been modified or removed in the RO, as previously described^{6, 37}. ROs resist infection by entire classes of phages without the need for phage receptor knock outs in general. This mechanism also does not require prior knowledge phages encountered in the facility. Specifically, modification or removal of one component of the translation machinery will impart some resistance to many classes of phages simultaneously, particularly, any phages that contain the forbidden codon. Importantly, many phages must undergo a large number of mutations to overcome each component of the RO's translation machinery that is modified or removed, which makes ROs quite stable for this purpose.

Modification or removal of additional translation machinery in the RO will both expand resistance to new classes of phages and increase resistance to classes of phages that the RO had already demonstrated some resistance to. Phages that did not contain forbidden codons initially, will now contain forbidden codons and will be unable to propagate efficiently within the RO. Phages that did contain forbidden codons initially will now contain additional forbidden codons and must undergo an increased number of mutations to overcome the additional missing or modified components of the RO's translation machinery. With sufficient modification or removal of translation machinery in the RO, the probability of a single phage overcoming this barrier by mutation becomes increasingly small.

In certain embodiments where a phage harbors its own tRNAs, these events can be countered using tightened recoding designs as described earlier, such that cells containing these phages will be quickly removed from the population. The RO can be engineered to include at least one restriction system or toxin-antitoxin system, wherein the methylase or antitoxin is absent and the restriction enzyme or toxin contains forbidden codons. In the basal state, the RO lacks unwanted forbidden codon activity and the at least one restriction enzyme or toxin are not active. If a phage infects the cell carrying its own tRNAs, the associated forbidden codons in the at least one restriction enzyme or toxin are expressed and any functional protein produced kills the cell.

It is understood that the term “phage resistance” is used herein to indicate that any aspect of the phage infection process, from the ability of the phage to contact and attach to the surface of the EO or REO to the ability of the phage to propagate throughout the EO or REO population, is impacted to any extent that can be measured. Sensitivity or resistance to phage can be tested using assays known in the art, including but not limited to: mean lysis time, plaque morphology assays, and burst size^{6, 37}. In specific embodiments, the EO or REO is tested against a panel of 15 phages, many of which commonly occur in bioreactors and impact culturing. Some exemplary phages in this list may include but are not limited to: Mu, λ cI857, M13, P1vir, P1 c1-100, MS2, phi92, phiX174, RTP, T1, T2, T3, T4, T5, T6, T7, ID11, 121Q, and Qbeta (Qβ). In certain embodiments, upon challenge with at least one type of phage in a phage infection assay, the titer of a phage produced from the EO or REO is reduced by at least 0.00001%, 0.001%, 1%, 5%, 10%, 20%, 30%, 40%, 50%, 60%, 70%, 80%, 90%, 91%, 92%, 93%, 94%, 95%, 96%, 97%, 98%, or 99% relative to the corresponding original organism (e.g., base strain). In certain embodiments, upon challenge with at least one type of phage in a phage infection assay, the titer of a phage produced from the EO or REO is reduced by at least 0.00001%, 0.001%, 1%, 5%, 10%, 20%, 30%, 40%, 50%, 60%, 70%, 80%, 90%, 91%, 92%, 93%, 94%, 95%, 96%, 97%, 98%, or 99% relative to the corresponding wild type organism or entity. In certain embodiments, a similar comparison can be made between the aforementioned entities, using other assays or a plurality thereof, as described or referenced herein, to determine if the EO or REO is phage resistant. In certain embodiments, assessment of phage resistance of the EO or REO is based on the collective analysis of all results collected from many assays, rather than a single one. In certain embodiments, phage resistance of the EO or REO is reasonably concluded as known to one skilled in the art, at the time.

Outbound HGT Blockage

Notably, if an RO is infected by phage and transduction occurs to carry the unwanted genetic material out of the RO and into a recipient organism, the recipient organism will be able to express the genetic material in most cases. Additionally, if the unwanted genetic material is carried out of the RO and into a recipient organism by a phage-independent mechanism, the recipient organism will also be able to express the genetic material in most cases. To address this, ROs can be further engineered to limit these types of HGT events.

Inbound HGT is naturally blocked by recoding an organism because certain components of the translation machinery are absent or modified that disable expression of the incoming genetic material. That said, recoded or nonrecoded genetic material can be expressed by nonrecoded recipient organisms because all machinery in the recipient should be present to allow expression of all codons and synonyms thereof. However, the RO itself can be further engineered via two additional steps, to avoid this: 1) the reduced genetic code of the RO can be exploited through a process called “codon expansion”, whereby forbidden codons are reintroduced into the RO's genetic material and assigned new meaning. 2) Subsequently, “codon encryption” can be performed on any amount of genetic material such that the products of the genetic material are only expressed properly in the RO and not by recipient organisms that might receive the genetic material. Notably, this can be done with any of the genetic material in the RO, genomic or non-genomic, and at any level, from one gene, to all genetic material in the organism. This process is described below as it relates to a transgene that was introduced into the RO for biomanufacturing, but is not meant to limit the invention in any way. By extension, similar embodiments can be drawn from this that involve other forms and any amount of genetic material in the RO (e.g., native genes, essential genes, etc.).

In these embodiments, for example, one or many forbidden codons can be inserted into the transgene of the RO. In this embodiment, codon expansion can occur through the introduction of an OTS that is expressed within the RO and that is specific for the forbidden codon and an NSAA, or through the introduction of an OTS that is expressed within the RO and that is specific for the forbidden codon and a standard amino acid. Alternatively an engineered tRNA of any kind can be used that recognizes the forbidden codon and inserts a standard amino acid, without the need of an introduced aminoacyl tRNA synthetase. A plurality of combinations can be used as well. Next, one of a few steps can be performed on the transgene for codon encryption: 1) a forbidden codon can be reassigned to encode an NSAA, 2) a forbidden codon can be reassigned to encode a standard amino acid that is not naturally inserted at the chosen site, 3) or a forbidden codon can be reassigned to encode the same standard amino acid that is naturally inserted at the chosen site. Sites for codon encryption should be carefully chosen such that the transgene products maintain functionality using the new code if the amino acid sequence is being changed. This is less critical if only the nucleic acid sequence is changed.

Clearly, it may be the case that phage resistance could be compromised if the OTS or engineered tRNA facilitate insertion of the associated amino acids at sites in the phage proteome that are tolerated by the phage and enable it to propagate. This situation can be avoided by using ROs with many different forbidden codons, some that are used for the purpose of phage resistance and some that are used for codon encryption. In these embodiments, the forbidden codons used for phage resistance would not be reassigned and the forbidden codons used for codon encryption would be reassigned. In this embodiment, even if the phage was able to use the codon encryption associated translation machinery (e.g., OTS) at some of its forbidden codons, the absence of translation machinery in the RO for its other forbidden codons would prevent its propagation. Furthermore, care should be taken if natural amino acids are used for codon encryption, where amino acids should be chosen such that the codon encryption associated translation machinery does not occur naturally in the environment, or has a low likelihood of occurring naturally in the environment. In this case, there is a low probability that the encrypted genetic material would be taken up by entities that could read it. If NSAAs that are synthetic (not naturally occurring) are used, the absence of these in addition to the associated OTSs in the open environment mean that this extra step described is less critical.

It is also useful to place transgenes or other engineered elements next to forbidden codon-containing toxins, using what is referred to herein as “linked masked toxins”. In this embodiment, the housekeeping genes and other potential regions of homology with genetic material of recipient entities are flanking the transgene and toxin and not in between. In this way, in the event of outbound HGT from this RO, the transgene will only be able to incorporate into the genome of the recipient entity by homologous recombination if the toxin gene is also incorporated, thereby killing the recipient and ridding this cell from the environment as an extra safety precaution should outbound HGT occur.

However, it is important to note that some embodiments described herein will specifically limit functional transfer of transgenes and engineered elements, but may have no effect on outbound HGT of housekeeping genes, etc. While codon encryption can be used throughout the genetic material of the EO or RO, in theory, as described herein, outward transfer of housekeeping genes is not expected to have deleterious environmental consequences, since such genes already generally are present in other entities in the environment.

Utility of Other Genome Designs

Inbound HGT Blockage

By way of background, restriction-modification systems normally found in bacteria include a restriction enzyme that recognizes a particular DNA sequence and makes a double-stranded cut in the DNA at or near that sequence, and also a methylase that recognizes the same sequence and introduces a methyl group on one or more of the bases in the sequence, such that the methylated DNA is resistant to recognition by the restriction enzyme. Typically, the recognition sequence of the restriction enzyme is four to eight bases and more typically fewer than eight), such that a bacterial genome of 4 million bases and 50% GC content will have many such sites. When a phage with normal and unmodified DNA infects such a host, the phage DNA will most frequently be cut and inactivated by the restriction enzyme, but in a small fraction of such infections the incoming DNA will first be modified by the methylase, and then phage replication can proceed. Similarly, when DNA from another bacterium is transferred into such a host, such DNA will generally be cut and then may be degraded into nucleotides and metabolized, but occasionally the incoming DNA will be modified by the methylase, and then incorporated into the genome to create a recombinant, hybrid organism.

As described herein, “super restricting genome designs” are those with additional features for limiting HGT. In this EO, all of the examples of a restriction site are removed from the EO's genome using editing methods or large replacement methods as described herein. Then, the corresponding restriction enzyme is expressed in the organism without the corresponding modification enzyme (e.g., methylase). The EO will not suffer from double-stranded breaks in its DNA because it lacks the associated recognition sequences. However, incoming DNA such as phage DNA or horizontally transferred DNA that possesses the restriction site will always be cut and such DNA will be unable to undergo modification to become resistant to cutting.

For example, according to the invention, a user can design a modified version of any bacterial genome that lacks the sequence GAATTC. The user can then express the EcoRI restriction enzyme in this host without EcoRI methylase. In an unmodified host such expression is generally lethal. The resulting host is then resistant to DNA phages and incoming HGT. In some embodiments, this genome can be combined with a recoded genome design to create an EO that is highly resistant to HGT.

Furthermore, in the construction of EOs, it is often necessary to modify the genome design in ways other than recoding, to enable a particular assembly method. For example, the enzymes LguI and BspQI recognize and cut the DNA sequence GCTCTTCN*NNN (i.e. these enzymes make a staggered cut outside the recognition sequence). It is therefore useful to eliminate such a restriction site from the designed genome, in order to use the enzyme in the preparation of component DNA fragments³⁸. As a result, it is often also convenient to construct EOs that are super-restricting.

Outbound HGT Blockage

A second type of linked masked toxin system can also be used in the context of a super restricting genome design to limit outbound HGT. In this embodiment, the restriction enzyme that lacks the methylase is the toxin. This will only be incorporated upon incorporation of the transgene or other engineered element that it is linked to, as described herein, and will be generally toxic when transferred into a recipient entity because the recipient entity's genome will have many sites cleaved by the restriction enzyme. This will serve to thereby kill the recipient entity and rid this cell from the environment as an extra safety precaution should outbound HGT occur.

Biocontainment

Uncontrolled Cell Growth

Unintended release of an EO or REO that biomanufactures a BP, into an open environment, poses significant risk to the open environment. It is understood that the open environment in this embodiment is that which is directly outside the culturing facility, as release into the environment where the REO is used, should be intentional.

For example, in the open environment just outside the facility where release should be unintentional, the EO or REO has the potential to propagate at a rate that may dominate or out compete specific native populations of entities in that open environment. Unintended release of EOs or REOs, even at low levels, has the potential to be catastrophic to open environments. Since such low level release may be unavoidable depending on culturing conditions and operations, this is becoming a significant risk in the culturing of REOs. Both extrinsic and instrinsic biocontainment mechanisms are needed to address this challenge.

While release into the environment where the REO is intended to be used, might be desired, the uncontrolled proliferation of the REO in that environment may not be desired. For example, if the open environment is the human gut, uncontrolled REO growth could be problematic if the REO is capable of outcompeting the native flora. It could be further problematic if outbound HGT occurs from the REO to the native flora.

Intrinsic biocontainment approaches have been more challenging to develop to date. Attempts to control cell growth have focused on essential gene regulation³⁹, inducible toxin switches⁴⁰, and engineered auxotrophies⁴¹. These approaches have been compromised by cross-feeding of essential metabolites, leaked expression of essential genes, or genetic mutations. Recent approaches have been developed^{10, 42} to address these challenges, that can be dramatically improved upon as described herein for the biomanufacturing of BPs within EOs and REOs and their release into open environments.

Utility of Recoded Genome Designs

ROs can be further engineered for biocontainment. In these embodiments, codon expansion is performed wherein at least one forbidden codon is re-inserted into at least one essential gene of the RO. In this embodiment, at least one OTS is expressed within the RO that is specific for the forbidden codon and at least one NSAA. Sites of forbidden codons should be carefully chosen to yield the respective functional essential protein products in the presence of the NSAA in the growth medium but not in the absence of it. It is understood that the essential gene protein product, by virtue of containing an NSAA, is different from a native protein product of the essential gene but is nevertheless functional. In this way, the RO's viability can be linked to the presence of the NSAA within the growth medium, as described previously¹⁰.

In certain embodiments, the log phase proliferation rate of the RO in the presence of the NSAA is greater than that in the absence of the NSAA by at least 2 fold, 3 fold, 4 fold, 5 fold, 6 fold, 7 fold, 8 fold, 9 fold, 10 fold, 15 fold, 20 fold, 25 fold, 30 fold, 40 fold, 50 fold, 100 fold, 200 fold, 500 fold, or 1,000 fold. In certain embodiments, the log phase doubling time of the RO in the presence of the NSAA is shorter than that in the absence of the NSAA by at least 2 fold, 3 fold, 4 fold, 5 fold, 6 fold, 7 fold, 8 fold, 9 fold, 10 fold, 15 fold, 20 fold, 25 fold, 30 fold, 40 fold, 50 fold, 100 fold, 200 fold, 500 fold, or 1,000 fold.

NSAA dependence or biocontainment using recoded genome designs is a powerful approach due to many features that can be tuned to confer a stable system. In some embodiments, essential genes can be chosen that can't be complemented by cross feeding of metabolites. In some embodiments, if an NSAA is chosen that does not occur in nature, leaky expression of target essential genes should be minimized. In some embodiments, mutation is minimized with more than one forbidden codon reinserted into essential genes, and more than one forbidden codon in any given essential gene. These modifications minimize the probability of mutation at the codon level, but select for mutation in trans. In some embodiments, additional modifications to the translation machinery (e.g., inactivation or deletion of redundant tRNAs that are not essential) or other cellular machinery can be made to enhance biocontainment and limit escape through mutations, as described previously¹⁰. These modifications enable a stable system whereby resulting strains exhibit undetectable escape frequencies upon culturing 10¹¹ cells solid media for 7 days or in liquid media for 20 days¹⁰.

Advanced recoding methods reported herein, will enable the creation of ROs whereby more than one forbidden codon has been partially or completely replaced with a synonymous codon, and the RO comprises a modification of more than one component of the cognate translation machinery (e.g., tRNA), be it deleted or engineered. In this embodiment, more than one forbidden codon can be reassigned in the RO, using more than one OTS, with specificities for distinct NSAAs not found in nature. The probability of escape using this system, and optionally, a plurality of other biocontainment mechanisms described herein, is expected to drop below that which we previously observed, to levels that will be well below what is required from a regulatory perspective to freely use these ROs for many applications. Collectively, if this RO or RRO is accidentally released from a closed environment, propagation and escape should be limited to an extent that it will be considered safe from a regulatory perspective. Additionally, in the event that release is intentional and biocontainment is a means by which growth can be regulated during the application, escape should be sufficiently low to permit its safe and stable application for this purpose, especially in a therapeutic context.

Notably, for applications that require a high level of safety, stability and control, a layered approach that combines HGT blockage and biocontainment, should be considered. For example, RROs, even with minimal recoding and without genome designs that could further restrict inbound HGT, are resistant to many phages. If the RRO is used as a living therapeutic within the gut where there are many phages, the RRO will have an significant competitive advantage. Additional modifications to enhance phage resistance of the RRO as described herein (e.g., additional recoding, super restricting genome designs, tightened recoding designs) will only increase this competitive advantage, further highlighting the need for controlled cell growth and a combined genome design that involves highly recoded organisms as well as biocontainment. We expect that these systems will be extremely optimized and enhanced for advanced living therapeutics applications and others applications involving open environments, as described herein.

In applications where RROs are used therapeutically, for example, within the gut, the orally delivered NSAA would need to maintain viability of the RRO during the application. Alternatively, the RRO can be cultured in the presence of the NSAA and released with a defined half life suitable for the therapeutic window. Further, this therapeutic window could be tuned with increasing numbers of RRO cells, the rate at which they're administered, or the concentration of the NSAA administered. NSAAs should be chosen that are not toxic and engineering to the OTS can be used to decrease the concentration of the NSAA required for the OTS to maintain RRO viability and the therapeutic dose.

Utility of Other Genome Designs

A recent study⁴² reported a layered biocontainment approach whereby mechanisms such as essential gene regulation and inducible toxin switches were individually optimized and combined into a single host strain. Similarly low escape frequencies (<1.3×10⁻¹²) were observed in this system. Notably, this biocontainment mechanism as well as a plurality of others could be combined with recoded genome designs (as described herein), into a single strain, to further limit escape to a level well below that which is considered safe from a regulatory perspective, especially for therapeutic applications.

NSAA Incorporation

Limited Protein Chemistries

Only twenty standard amino acids are encoded from 64 codons, due to the redundancy of the genetic code. There is a need to produce polypeptides and proteins with expanded chemistries. Cofactors have evolved alongside proteins to make up for the lack of chemistries that exist amongst the twenty standard amino acids. Higher organisms have evolved post-translational modification to increase the diversity of amino acid side chains further. Artificial approaches have also been developed such as protein modification in vitro. While bacteria would be a preferred host for many living therapeutics applications, for example, there remains a need for methods of biomanufacturing polypeptides and proteins using expanded chemistries in this host.

Utility of Recoded Genome Designs

For applications where expanded chemistries are desired for incorporation into BPs, ROs can be engineered for NSAA incorporation into polypeptides and proteins. In this case, a protein can be designed to contain an NSAA at a specific location to impart a desired property to it.

In these embodiments, ROs can be useful for NSAA-containing protein or polypeptide production. In certain embodiments, the protein containing the NSAA is more stable than a corresponding wild type protein. In certain embodiments, a protein containing an NSAA has a functional property (e.g., enzymatic activity) that is absent in the corresponding wild type protein. In certain embodiments, the protein containing the NSAA only has a chemical handle that enables binding or chelation (e.g., as opposed to altered protein folding). In certain embodiments, the NSAA allows the protein to fold in a specific way as to impart new enzymatic activity.

Codon expansion is performed in the RO where at least one forbidden codon is inserted into at least one transgene in the RO. Sites of forbidden codons are carefully chosen to yield the transgene product with the desired properties. In this embodiment, an OTS is expressed within the organism that is specific for the forbidden codon and an NSAA. In this embodiment, if the NSAA is included within the growth medium, the at least one transgene product will result from the incorporation of the NSAA into the protein product, as described previously for ROs^{6, 14}. This process can result in biomanufacturing of proteins with NSAAs that have expanded chemistries in bacteria, which proliferate and produce the target protein with high efficiency. In certain embodiments, NSAAs can be chosen that are especially low in cost and ROs can also be evolved to use very low concentrations of the NSAA, reducing the cost of production further.

Notably, ROs with a plurality of forbidden codons that are either partially or completely replaced with synonymous codons in the RO, could significantly enhance these applications. This would enable insertion of many different NSAAs in the same cell, enabling a diverse array of additional chemistries beyond the standard twenty, to be inserted into proteins.

Utility of Other Genome Designs

It is understood that ROs are not required for NSAA incorporation into polypeptides and proteins in an EO^{6, 7, 27}. These embodiments suffer from competition of translation machinery at forbidden codons in most cases. For example, in the case of an EO, if the forbidden codon meant to encode an NSAA is inserted into a transgene in the presence of an EO with an OTS, the OTS will insert the NSAA at forbidden codons throughout the native proteome and the native translation machinery will insert the native amino acid (or terminate translation, in the case of a release factor) at the forbidden codons in the transgene. Ultimately these embodiments suffer from poor yield of the target transgene product whereby a lot of it is either truncated or contains an undesired standard amino acid. Yield also suffers as a result of poor EO fitness as a large percentage of the native genes aren't properly expressed with the NSAA inserted. Therefore, ROs are a better platform for this purpose.

Generation of EOs

To generate an EO with a target genome design that confers a specific functional property, an in silico design phase may be implemented. It is often challenging to isolate the target genome design in silico that will impart viability to the organism, let alone the specific functional property. Often, one genome design is drafted in silico, and this design is then built from a wild type entity in the laboratory and tested for function. This process is highly inefficient in terms of time and cost because design rules are insufficiently understood to be able to choose a design in silico that is likely to work in the build phase. The subsequent build process will thus involve iterating laboriously through the errors (herein referred to as “debugging”), such that the larger the number of changes desired, relative to the wild type ancestral entity, the longer the “debugging” process will take, making the process extremely unscalable.

Advanced approaches for building EOs with genome designs consisting of many genomic changes as described herein, are desperately needed in the field. This need will further increase as the field of synthetic biology matures and additional applications for EOs come to market. Many of these applications require EOs with functional properties imparted by genome designs that contain a large number of modifications. For example, advanced. applications of EOs will likely require functional properties such as controlled viability and HGT blockage for release into open environments (e.g., living therapeutics), or NSAA incorporation to produce highly advanced BPs for biomanufacturing (e.g., products with complex properties).

An approach to building EOs in a scalable process that enables one to install many changes to the genome efficiently, should pair 1) better genome design rules with 2) increased efficiency of genome modification methods. The first part of this approach would impart necessary in silico predictive power with which to be able to sort through genome designs that are unlikely to work (either due to viability or lack of imparting the functional property), enriching the library of designs that are actually built during the build phase, for those that are more likely to work. The second part of this approach would then enable efficient iteration through the enriched library. To date, there has been no such approach that efficiently combines these two components.

Methods of Generating EOs

The generation of an EO is carried out via one or more design-build-test (DBT) cycles that can involve editing the genome via many small changes, herein referred to as “editing methods”, or replacement of large native fragments of the genome with synthesized fragments via fewer total changes, herein referred to as “large replacement methods”.

In some embodiments, the EO comprises genetic material that is both genomic and non-genomic and the methods described herein also apply to these embodiments. In some embodiments, the synthesized fragment used for replacement can be double stranded. In some embodiments, the synthesized fragment used for replacement can be single stranded⁴³. In some embodiments, a plurality of types of synthesized fragments are used.

Editing methods and large replacement methods can be used individually or in combination in any organism (e.g., species and strains). In some embodiments, a plurality of methods can be used in an organism. In some embodiments, specific components of these methods and the described processes may vary for different organisms.

In some embodiments, generation of the functional property is directly or indirectly selectable. In some embodiments, the functional property is neither directly nor indirectly selectable. In some embodiments, a screen must be used. In some embodiments, generation of the functional property will require that a plurality of selection and screening methods are used. In some embodiments, high throughput screening is used. In some embodiments, liquid handling and automation are used. In some embodiments, a plurality of these approaches are used.

Editing methods can be used such that many edits are introduced in parallel. Large replacement methods can be used such that many synthesized fragments (containing many edits) are introduced in parallel. These embodiments are herein referred to as “pooled methods”. In some embodiments, a plurality of pooled methods may be used.

In some embodiments, pooled editing methods can involve many different edits targeting the same site or region of the genome. In some embodiments, pooled editing methods can involve many different edits targeting different sites or regions of the genome. In some embodiments, pooled large replacement methods can involve many different synthesized fragments (containing many different edits) targeting the same site or region of the genome. In some embodiments, pooled large replacement methods can involve many different synthesized fragments (containing many different edits) targeting different sites or regions of the genome. In some embodiments, a plurality of the above methods can be used for a single EO.

Nucleic acid sequence data can be associated with the presence or absence of experimental data in terms of the functional property or viability. In some embodiments, a plurality of associations can be made. These nucleic acid sequence data can be generated by sequencing all nucleic acid sequences generated during the experiment, or barcodes associated with pre-determined sequences. The absence of certain sequence data or relative abundance of certain sequence data can also be used to gather both negative and positive data., increasing the abundance of data collected. These data can be generated using a plurality of methods across pooled editing methods, non-pooled editing methods, pooled large replacement methods, and non-pooled large replacement methods. Over time, the abundance of nucleic acid sequence data associations can be used to inform partial or full genome designs that will or will not generate the desired functional property, viability, or both. This will serve to reduce the time and cost associated with EO generation, as genome design library sizes should decrease over time. As this happens, the efficiency of editing and large replacement methods is also expected to increase. In some embodiments where non-genomic material is modified, the same approach can be applied. In some embodiments, training data can be generated from these experiments and associations made, using a ML-assisted approach as is described further herein.

Design

An in silica stage is used to generate genome designs of interest that could lead to a desired functional property. In some embodiments, only some parts of the genome are modified relative to the ancestral entity. In some embodiments, only one genome design is used, and in others, many genome designs are used. In some embodiments, a single genome design can impart a plurality of functional properties.

For large replacement methods, DNA that is used to build the design or designs can involve double stranded DNA fragments up to 200,000 bp in size. Fewer synthesized fragments will require fewer steps toward assembly. In some embodiments, much larger fragments can be used. In some embodiments, much smaller fragments can be used. In some embodiments, even for large replacement methods, single stranded DNA oligonucleotides “oligos” can be used containing the long sequence to be integrated as previously reported^{43, 44}. For editing based methods, single stranded DNA oligos are used that can make all desired single edits in the ancestral entity.

If many genome designs are being analyzed for a single outcome. DNA can be ordered for all designs concurrently. In this embodiment, DNA targeting the same region of the genome but with different designs, can barcoded and pooled during the build stage. In this embodiment, only target designs will yield viable or functional cells, or both, in the build stage. Sequencing the library of resulting barcodes in the population, or other regions of the DNA directly, can be used to associate viable cells or cells with the functional property with the associated designs. In the case where only viability is being screened for, or a selection is linked to the functional property, or both, then non-viable cells (and associated designs) should drop out of the population. In these embodiments, the absence of barcodes or specific sequences can be used to inform negative data.

In some embodiments, if many genome designs are used, data can be generated for a given native fragment (large replacement methods) or single site within the genome (editing based methods) as to which designs are viable versus inviable or impart the functional property versus do not impart the functional property. Many data points can be collected this way. In some embodiments, modeling or ML-assisted approaches can then be used to learn from these data to inform better future designs in which fewer synthesized fragments will be necessary during future EO generation projects, lowering the cost and reducing the overall time toward EO generation over time.

Build

The build phase starts with introducing DNA containing the synthesized fragments or oligos, into the cell. In some embodiments this can be done via transformation, electroporation, transduction (e.g., P1), or conjugation. In some embodiments, for large replacement methods, the synthesized fragments are contained within an episome or BAC. In some embodiments, for large replacement methods, the synthesized DNA to be incorporated is anywhere from 1,000 bp to 200,000 by in size. In some embodiments, oligos can be produced within the entity, in vivo⁴⁵, as previously described. In some embodiments, much larger fragments can be used. In some embodiments much smaller fragments can be used.

Homologous recombination is used to facilitate incorporation of synthesized DNA fragments or oligos⁴³ into the target region of the genome. In some embodiments, recombination is assisted by a recombinase introduced into the cell such as, for example, Lambda Red^{46, 47}. In some embodiments, genetic modifications can be made to the entity to enhance recombination efficiency. For large replacement methods, in some embodiments where an episome or BAC is used, CRISPR is used to linearize the species to expose the homologous arms for integration at the target site. In some embodiments, the integration includes an antibiotic resistance gene or other selectable marker. For editing methods, in some embodiments where oligos are introduced in pools, Multiplex Automated Genome Engineering (MAGE) is used, as described previously⁴³. In some embodiments, genetic modifications can be made to the entity to enhance recombination efficiencies. For editing methods, in some embodiments, certain components of the entity's mismatch repair machinery (e.g., mutS, mutL), are modified to enhance retention of desired edits. For editing methods, in some embodiments, co-selection is used to increase the efficiency of MAGE as previously described⁴⁸. For editing methods, in some embodiments, CRISPR can be used to eliminate non-edited cells from the population⁴⁹, increasing the efficiency of the build process.

Many iterations of DNA introduction followed by recombination are applied to replace the desired regions of the genome with synthesized DNA. In some embodiments, the entire genome is replaced with synthesized DNA. There are many variations of iterative assembly that have been described previously^{3, 5, 6, 13}. In some embodiments, iterations are done sequentially in a single entity. In some embodiments, the genome is split into pieces across many entities and iterations are done on many entities in parallel and the partial genomes hierarchically merged after iterative building is complete. In some embodiments, hierarchical merging of partial genomes can be done via conjugation, for example.

Test

Testing can occur at many phases, both throughout the build cycle and at the end of it. The earliest test phase occurs throughout the build phase. During the build phase, populations of cells exposed to one or many synthesized fragments or oligos are assessed for viability or the functional property, or both, which constitutes an important test to determine if the genome design was a successful one. Viable cells or those with the functional property, or both, are then further screened for the synthesized fragment or incorporation of the desired edit, via sequencing and PCR, which constitutes an additional test to confirm that the cell contains the synthesized fragment at the desired location. After the build phase is complete, additional testing is performed at the level of sequencing and PCR to ensure that the resulting EO contains synthesized fragments or desired edits at all desired locations and to verify general genomic integrity at the level of background mutation accumulation, etc.

In some embodiments where many designs are pooled, throughout the build cycle, a screen can be done on the population of viable cells for the functional property of the associated genome design, ultimately yielding both viable and Functional cells. In some embodiments, a selection can be linked to the functional property of the associated genome design, ultimately yielding both viable and Functional cells as well. In some embodiments, both methods can be used. In some embodiments, one or both methods can be used during the build phase to reduce the number of DBT cycles.

Throughout the build cycle, viability or presence of the functional property, or both, are screened for. In general, pooled genome designs are meant to minimize the number of DBT cycles and “debugging” such that many designs are analyzed in parallel. As mentioned previously, coupled with this improvement, ML-assisted approaches that learn from these data (generated from pooled or unpooled data or both) can further inform future genome design efforts, which will minimize the number of genome designs analyzed for a given EO generation project, increasing the efficiency of this process over time.

ML-Aided Genome Design Coupled with Library-Based Methods for Building Many Genomes at Once

In general, if many changes are to be made to a wild type ancestral entity, to isolate a target genome with a design that imparts all desired functional properties, a process that allows many changes to be made at once is going to be more efficient. Large replacement methods are typically better for this reason because they allow for the insertion of large synthesized fragments of DNA that comprise large stretches of modifications as outlined in the genome design. Editing methods are in some cases, slower, because modifications must be made one at a time. While pooling many changes is useful, this is only true up to a certain number of changes, as the probability of finding a single entity in the population containing all modifications drops, as the number of introduced modifications increases.

However, while large replacement methods are theoretically faster, in practice, they can be slower, if the design rules that are used to predict the nucleic acid sequence of the synthesized fragments, have weak predictive power in terms of the resulting viability or functional property or both. In practice, often, a given synthesized fragment will not generate a viable cell upon integration into the genome, due to a number of nonviable design components in the fragment, that are difficult to isolate. Alternatively, a given synthesized fragment may not generate a functional cell upon integration into the genome, due to a number of nonfunctional design components in the fragment, that are difficult to isolate. In some instances, both are true. The debugging process of finding the faulty components typically takes much too long, completely canceling out the time savings that large replacement methods promise. An approach using the aforementioned processes, whereby many different synthesized fragments representing a given region of the genome but derived from many different genome designs, are pooled in a single cell, has an advantage over a non-pooling large replacement method because it would eliminate this problem. This approach further has the ability to generate a tremendous amount of data necessary to enable a ML-assisted approach to generating highly predictive genome design rules. These rules can be strengthened overtime, minimizing the number of genome designs that are pooled for a given EO generation project.

Machine Learning Methods for Improvement of Genome Designs

As described above, genome designs are tested by large replacement and/or editing methods. These genome designs are collected and analyzed using machine learning (ML) approaches to develop a machine learning model. The trained machine learning model is useful for informing future designs, thereby reducing the time and cost associated with testing and generating further EOs.

In preferred embodiments, a machine learning model is trained to generate a prediction indicating whether a recoded organism, with one or more edits in the genome, is likely to be a functional organism. As used herein, the term “functional organism” (e.g., including “functional recoded organism” and “functional engineered organism”) refers to an organism that has at least one functional property as described herein. In particular embodiments, the machine learning model receives, as input, a combination of edits to a genome and the genomic locations in which the edits are located, and outputs a prediction of whether a recoded organism with the combination of edits at those genomic locations is likely to be a functional recoded organism or a non-functional recoded organism. Notably, the application of this toward a recoded genome design was used as an example and is not meant to limit the invention in any way. An analogous process as described herein, can be used to determine the edits associated with any genome design, or combinations of genome designs that can be used to generate any functional property or combinations of functional properties, or simply viability alone. In some embodiments, a prediction indicates whether an engineered organism, with one or more edits in the genome, is likely to be a functional organism (e.g., have the at least one functional property) and a viable functional organism.

In various embodiments, the machine learning model is any one of a regression model (e.g., linear regression, logistic regression, or polynomial regression), decision tree, random forest, support vector machine, Naïve Bayes model, k-means cluster, or neural network (e.g., feed-forward networks, convolutional neural networks (CNN), or deep neural networks (DNN)). The machine learning model can be trained using a machine learning implemented method, such as any one of a linear regression algorithm, logistic regression algorithm, decision tree algorithm, support vector machine classification, Naïve Bayes classification, K-Nearest Neighbor classification, random forest algorithm, deep learning algorithm, gradient boosting algorithm, and dimensionality reduction techniques. In various embodiments, the machine learning model is trained using supervised learning algorithms, unsupervised learning algorithms, semi-supervised learning algorithms (e.g., partial supervision), weak supervision, transfer, multi-task learning, or any combination thereof. In various embodiments, the machine learning model comprises parameters that are tuned during training of the machine learning model. For example, the parameters are adjusted to minimize a loss function, thereby improving the predictive capacity of the machine learning model.

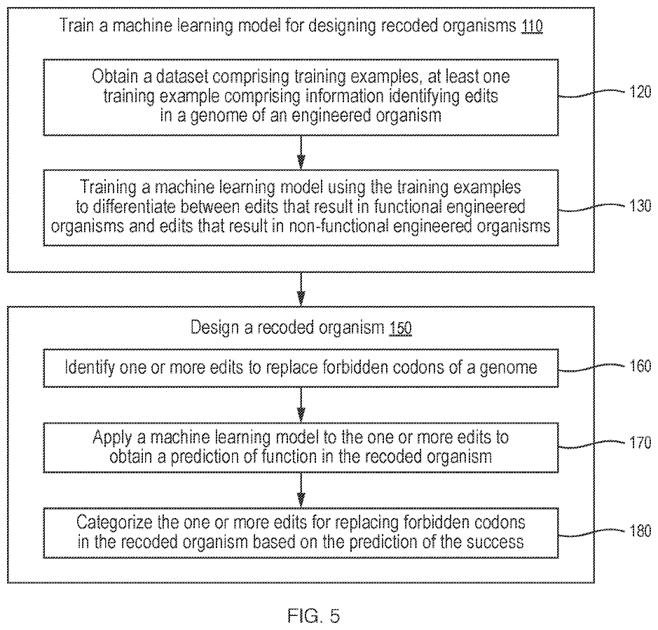

FIG. 5 depicts a flow diagram for training and deploying a machine learning model for designing a recoded organism.

Step 110 in FIG. 5 involves training a machine learning model for designing recoded organisms 110. The training of the machine learning model involves steps 120 and step 130. Step 120 involves obtaining a dataset comprising training examples that are used to train the machine learning model. At least one of the training examples includes information identifying edits in a genome that were made to a previously engineered organism. In various embodiments, each training example in the dataset corresponds to a previously engineered organism containing one or more edits across the genome.

The term “obtaining a dataset” encompasses obtaining an engineered organism and performing one or more assays on the engineered organism to obtain the dataset. As one example, the previously engineered organism can undergo assaying and sequencing to generate sequencing data that reveals the sequence of the organism's genome. In various embodiments, the term “obtaining a dataset” encompasses engineering the organism (e.g., by incorporating one or more edits in the organism) and performing one or more assays on the engineered organism. The one or more edits across the genome of the engineered organism can be made using large replacement methods or editing methods. Additionally, the term “obtaining a dataset” encompasses receiving, from a third party, a dataset identifying edits in the genome. In such embodiments, the third party may have performed the assay and sequenced the organism's genome to generate the dataset.

Step 130 involves training the machine learning model using the training examples. Generally, the machine learning model is trained to differentiate between one or more edits that result in a functional engineered organism and one or more edits that result in a non-functional engineered organism. For example, the machine learning model is trained to recognize patterns across the training examples that contribute towards a functional or non-functional engineered organism. As a specific example, the machine learning model is trained to identify particular genomic locations that, if edited, likely cause an engineered organism to be non-functional. As another specific example, the machine learning model can be trained to identify particular genomic locations that, if edited, result in an engineered organism that is functional.

In various embodiments, each training example corresponds to a previously engineered organism. In various embodiments, a training example identifies one or more of the following elements: 1) edits in the genome of the engineered organism, 2) positions of the edits in the genome, and 3) a reference ground truth indicating whether the engineered organism was a functional engineered organism or a non-functional engineered organism. In various embodiments, a training example includes all three of the aforementioned elements that correspond to an engineered organism.

In various embodiments, edits in the training example can refer to a combination of edits throughout the genome accomplished using editing methods, as described above. For example, the combination of edits in the training example can refer to the replacement of a group of codons (e.g., group of forbidden codons) at locations in the genome. Such combination of edits can be synonymous codons for replacing forbidden codons. In various embodiments, edits in the training example refer to a replacement nucleic acid fragment that replaces a reference region of the genome, as described above in relation to the large replacement method. For example, the edits in the training example can refer to a nucleic acid fragment at least 100,000 nucleotide bases in length that replaced a reference region at a particular location of the genome. In some embodiments, edits in the training example can refer to a combination of edits within a replacement nucleic acid fragment that replaces a reference region of the genome accomplished through large replacement methods. For example, edits in the training example can be a combination of edits that replace a group of codons (e.g., a group of forbidden codons) the reference region of the genome. In various embodiments, edits in the training example can refer to both edits accomplished through editing methods as well as edits in replacement nucleic acid fragments accomplished through large replacement methods. In some embodiments, each training example has at least 100 edits. In some embodiments, each training example has at least 200, 300, 400, 500, 600, 700, 800, 900, or 1000 edits. In some embodiments, each training example has at least 10⁴, 10⁵, or 10⁶ edits.

In various embodiments, the position of the edits in the genome refer to a particular location or a range of locations in the genome. For example, the position of the edits can identify a base position or a range of base positions on a chromosome. In various embodiments, the position of the edits can identify one or more of a chromosome, an arm (e.g., long arm or short arm) of the chromosome, a region, a band (e.g., a cytogenic band labeled as p1, p2, p3, q_{1,} q2, q3, etc.), a sub-band, and/or a sub-sub-band. An example of such a position can be denoted as 7q31.2 which refers to chromosome 7, the q-arm, region 3, band 1, and sub-band 2.

The reference ground truth of the training example provides an indication as to whether the corresponding previously engineered organism was a functional or non-functional engineered organism. In various embodiments, the reference ground truth can be a binary value. For example, a value of “1” indicates that the engineered organism was a functional engineered organism whereas a value of “0” indicates that the engineered organism was a non-functional engineered organism. In various embodiments, the reference ground truth can be a continuous value. The continuous value provides a measure of the function of the engineered organism. As an example, the reference around truth can be a value between “0” and “1,” where a value closer to “1” indicates that the organism exhibits improved viability in comparison to the viability of a different organism with a value closer to “0.” As another example, the reference ground truth can be a percentage (e.g., between 0 and 100%) that represents the percentage viability of organisms with the particular combination of edits at locations across the genome.

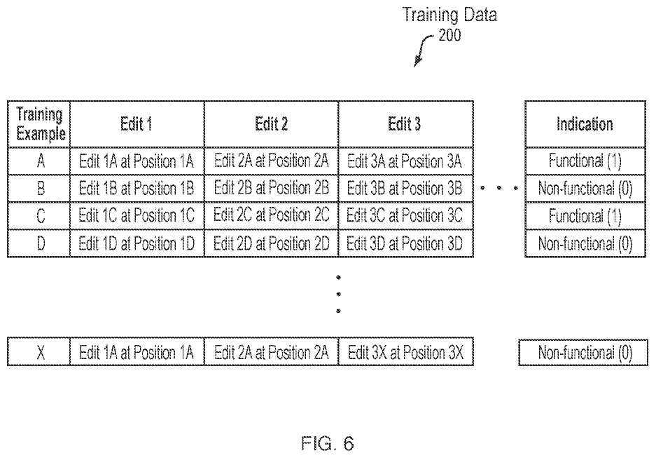

Reference is now made to FIG. 6, which depicts example training data used to train the machine learning model, in accordance with an embodiment. The training data 200 includes individual training examples that correspond to previously engineered organisms. As shown in FIG. 6, each training example (e.g., each row of training data 200) identifies a combination of edits at different positions across the genome of an engineered organism. The combination of edits replace a group of codons (e.g., group of forbidden codons) at the different positions across the genome. Although FIG. 6 only depicts three edits for each training example, in various embodiments, each training example may have hundreds, thousands, or even millions of edits that were previously engineered in the organism. Additionally, FIG. 6 depicts several different training examples (e.g., training examples A, B, C, D, and X); however, in various embodiments, there may be more training examples in the training data 200 for training the machine learning model.

Referring to “Training Example A” in FIG. 6, an engineered organism has an Edit 1A at Position 1A in the genome, an Edit 2A at Position 2A in the genome, an Edit 3A at Position 3A in the genome, and so on. This particular engineered organism was a functional engineered organism. Therefore, the training example includes an indication (as documented in the final column) of viability, which in this example is a binary value of “1.” Referring to “Training Example B” in FIG. 6, an engineered organism has an Edit 1B at Position 1B in the genome, an Edit 2B at Position 2B in the genome, an Edit 3B at Position 3B in the genome, and so on. This particular engineered organism was a non-functional engineered organism and therefore, the training example includes an indication (as documented in the final column) of non-viability, which in this example is a binary value of “0.” Training Examples C, D, and X are similarly organized in the training data 200.

In various embodiments, different training examples may have a subset of common edits across the genome at common positions. For example, in FIG. 6, Training Example A may have common edits at common positions in relation to the edits for Training Example X. Both Training Example A and Training Example X have an Edit 1A at Position 1A and an Edit 2A at Position 2A. However, the training examples differ at a third edit, where Training Example A has Edit 3A at Position 3A whereas Training Example X has Edit 3X at Position 3X. Additionally, Training Example A includes a reference ground truth of functional (1) whereas Training Example X includes a reference ground truth of non-functional (0). Having training examples that have subsets of common edits across the genome at common positions enables the training of the machine learning model to identify patterns, such as edits at particular positions in the genome, that likely cause a functional or non-functional engineered organism. Thus, the machine learning model can learn that the third edit of Training Example X (e.g., Edit 3X at Position 3X) may contribute towards a non-functional engineered organism given that the first and second edits were in common with a functional engineered organism (e.g., Training Example A).

Returning to FIG. 5, step 150 involves designing a recoded organism by applying the machine learning model that is trained to generate a prediction indicating whether a recoded organism, with one or more edits in the genome, is likely to be a functional recoded organism. As shown in the embodiment depicted in FIG. 5, step 150 of designing a recoded organism includes steps 160, 170, and 180.

Step 160 involves identifying one or more edits for replacing forbidden codons of a genome. In various embodiments, the one or more edits include at least 100 edits. In various embodiments, the one or more edits include at least 200, 300, 400, 500, 600, 700, 800, 900, or 1000 edits. In some embodiments, the one or more edits include at least 10⁴, 10⁵, or 10⁶ edits. In one embodiment, the gene edits are individual replacement edits to a group of forbidden codons located at different positions of the genome. In one embodiment, the gene edits are large replacement nucleic acid fragments that replace a reference region of the genome. Such large replacement nucleic acid fragments may include replacement edits to a group of forbidden codons that are located within the reference region of the genome. In one embodiment, the gene edits are a combination of individual replacement edits and large replacement nucleic acid fragments that replace a forbidden at different positions across the genome.

Step 170 involves applying the trained machine learning model to edits to obtain a prediction of the functionality of the recoded organism. In one embodiment, applying the trained machine learning model may involve providing the edits identified at step 160 as input to the trained machine learning model. In various embodiments, applying the trained machine learning model involves providing positions across the genome (e.g., positions of forbidden codons) that the edits identified at step 160 are to inserted. In various embodiments, applying the trained machine learning model involves providing, as input, both 1) the edits identified at step 160 and 2) the positions across the genome that the edits are to be inserted to the machine learning model. The machine learning model outputs a prediction that is informative of the functionality of the recoded organism that includes the inputted edits. Specifically, given that the machine learning model has been trained to distinguish between edits that are likely to cause a functional or non-functional engineered organism, the machine learning model can output a prediction as to whether this particular combination of edits located at positions of the genome is likely to lead to a functional or non-functional engineered organism.

In various embodiments, the machine learning model can output a predicted score that is indicative of whether the recoded organism with the edits at particular locations in the genome would likely lead to a functional or non-functional recoded organism. For example, the score may be a value between 0 and 1, thereby representing a probability that the recoded organism is likely to be a functional recoded organism.

At step 180, based on the prediction outputted by the machine learning model, the identified edits at particular locations of the genome are categorized. As an example, the identified edits can be categorized as candidate edits that are to be further tested and validated. Such candidate edits can be tested in vitro by engineering a recoded organism to have the candidate edits using editing or large replacement methods, as described above. As another example, the identified edits can be categorized as non-candidate edits. Such non-candidate edits need not be subsequently tested or validated.

In various embodiments, the identified edits are categorized using predicted score outputted by the machine learning model. As one example, identified edits that are assigned a score above a threshold value are categorized as candidate edits for further testing. In various embodiments, the threshold score is 0.5, 0.6, 0.7, 0.75, 0.8, 0.85, 0.90, 0.91, 0.92, 0.93, 0.94, 0.95, 0.96, 0.97, 0.98, or 0.99. Identified edits that do not satisfy the threshold score criterion are categorized as non-candidate edits.

Altogether, the implementation of the machine learning model enables in silico prediction and categorization of edits that can be rapidly screened out. Thus, only candidate edits are used in genomic designs for further testing whereas non-candidate edits are removed from further consideration. This eliminates the need to test all combinations of edits in vitro which is significantly time-consuming and costly.

Computing Device

The methods described above, including the methods of training and deploying a machine learning model for designing a recoded organism, are, in some embodiments, performed on a computing device. Examples of a computing device can include a personal computer, desktop computer laptop, server computer, a computing node within a cluster, message processors, hand-held devices, multi-processor systems, microprocessor-based or programmable consumer electronics, network PCs, minicomputers, mainframe computers, mobile telephones, PDAs, tablets, pagers, routers, switches, and the like.

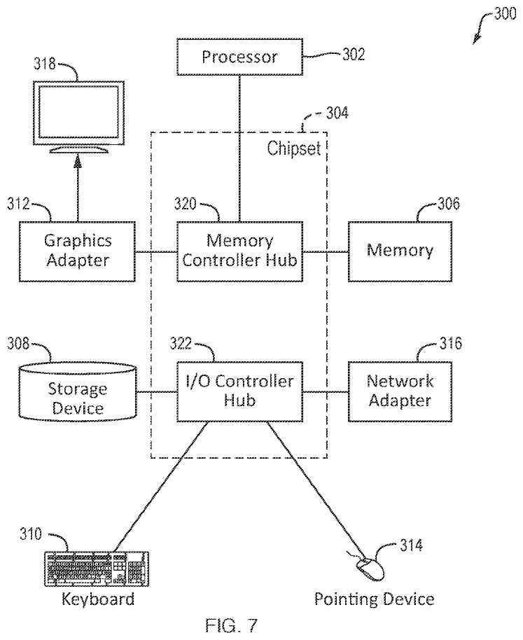

FIG. 7 illustrates an example computing device 300 for implementing the methods described above in relation to FIGS. 5 and 6. In some embodiments, the computing device 300 includes at least one processor 302 coupled to a chipset 304. The chipset 304 includes a memory controller hub 320 and an input/output (I/O) controller hub 322. A memory 306 and a graphics adapter 312 are coupled to the memory controller hub 320, and a display 318 is coupled to the graphics adapter 312. A storage device 308, an input interface 314, and network adapter 316 are coupled to the I/O controller hub 322. Other embodiments of the computing device 300 have different architectures.

The storage device 308 is a non-transitory computer-readable storage medium such as a hard drive, compact disk read-only memory (CD-ROM), DVD, or a solid-state memory device. The memory 306 holds instructions and data used by the processor 302. The input interface 314 is a touch-screen interface, a mouse, track ball, or other type of input interface, a keyboard, or some combination thereof, and is used to input data into the computing device 300. In some embodiments, the computing device 300 may be configured to receive input (e.g., commands) from the input interface 314 via gestures from the user. The graphics adapter 312 displays images and other information on the display 318. For example, the display 318 can show an indication of a treatment, such as a treatment validated by applying the cellular disease model. As another example, the display 318 can show an indication of a common chemical structure group likely contributes toward an outcome e.g., favorable outcome or adverse outcome). As another example, the display 318 can show a candidate patient population that, through implementation of the cellular disease model, has been predicted to respond favorably to an intervention. The network adapter 316 couples the computing device 300 to one or more computer networks.

The computing device 300 is adapted to execute computer program modules for providing functionality described herein. As used herein, the term “module” refers to computer program logic used to provide the specified functionality. Thus, a module can be implemented in hardware, firmware, and/or software. In one embodiment, program modules are stored on the storage device 308, loaded into the memory 306, and executed by the processor 302.

The types of computing devices 300 can vary from the embodiments described herein. For example, the computing device 300 can lack some of the components described above, such as graphics adapters 312, input interface 314, and displays 318. In some embodiments, a computing device 300 can include a processor 302 for executing instructions stored on a memory 306.

Non-Transitory Computer Readable Medium

Also provided herein is a computer readable medium comprising computer executable instructions configured to implement any of the methods described herein. In various embodiments, the computer readable medium is a non-transitory computer readable medium. In some embodiments, the computer readable medium is a part of a computer system (e.g., a memory of a computer system). The computer readable medium can comprise computer executable instructions for training or deploying a machine learning model for determining whether edits are likely to lead to a functional or non-functional recoded organism.

Generation of REOs

The REO is generated by introducing the at least one additional nucleic acid sequence or modification to make the organism fully proficient for biomanufacturing of the at least one BP. Importantly, where the REO is a RRO, if the additional genetic material is to be expressed as a protein or polypeptide within the RRO, it is important that this additional genetic material is recoded. For example, if the additional genetic material is an episome with a resistance gene, forbidden codons should be removed from the resistance gene. As another example, if the additional genetic material is a transgene encoding the BP where the BP will be expressed in the RRO, forbidden codons should be removed from the transgene.

In certain embodiments, the REO comprises more than one additional or modified nucleic acid sequence or element relative to the EO. In some embodiments, the process of generating the final REO includes a plurality of methods described herein for the generation of EOs. Notably, in some embodiments, where possible, transgenes, exogenous genetic material and other genetic material that are particularly risky to share with native organisms or entities in an open environment or the culturing facility, should be genomically integrated to further avoid undesired HGT to other entities in that environment. During the build or test phases, final REO performance is assessed using assays that vary depending on the BP that is manufactured and the functional property of the EO. In certain embodiments, final REO performance should exhibit characteristics of both the EO and the base strain. In certain embodiments, a mouse model can be used to confirm that the functional property and optimization for the open environment is sufficient to impart the desired therapeutic outcome in the subject.

Culturing and Production of REOs

The REOs that can be made according to the invention are unlimited in purpose. They can be used as medicines (e.g., living therapeutics, living vaccines), research tools (e.g., use of living therapeutics or living vaccines for research or diagnostic use), food products (e.g, probiotics, ingredients), or environmental tools (e.g., bioremediation). Use of the REO may be by any means suitable.

The REOs disclosed herein are useful for biomanufacturing of BPs and their release into open environments by methods known in the art. For example, in an aspect, the present disclosure provides a method of producing an REO, the method comprising culturing an REO under suitable conditions. In some embodiments the conditions may be anaerobic. In some embodiments the conditions may be aerobic.

The REO may be cultured by batch fermentation, fed-batch fermentation, or continuous fermentation. The cells of the REO may be cultured in suspension or attached to solid carriers in shaker flasks, fermenters, or bioreactors. The culture medium may contain buffer, nutrients, NSAAs, standard amino acids, oxygen, inducers, other additives, and optionally selective agents (e.g., antibiotics). In certain embodiments, the culture medium can contain one, all or a combination of any of these components. Where expression of the transgene is inducible, such that the cells are not burdened with protein production at the proliferation phase, inducers for the transgene expression can be added between the proliferation phase and the protein production phase. Exemplary fermentation processes are disclosed, for example⁵⁰⁻⁵². After fermentation, the cells and supernatant can be harvested and the BP can be isolated and purified from the proper fraction using methods known in the art.

The REOs that can be cultured according to the method disclosed herein, can be made with cGMP conditions (as referenced herein: https://www.fda.gov/drugs/pharmaceutical-quality-resources/current-good-manufacturing-practice-cgmp-regulations) or non-cGMP conditions, such as research grade. In certain embodiments, the entity, EO, or REO are suitable for cGMP manufacturing. In certain embodiments all of the entity, EO, or REO are suitable for cGMP manufacturing.

Uses of REOs

The uses for REOs made according to the invention are unlimited in purpose. They can be used as medicines (e.g., living therapeutics, living vaccines), research tools (e.g., use of living therapeutics or living vaccines for research or diagnostic use), food products (e.g, probiotics, ingredients), or environmental tools (e.g., bioremediation). Use of the REO may be by any means suitable.

The BPs that can be made within the REO according to the invention are unlimited in purpose. They can include but are not limited to: nucleotides, nucleic acids, amino acids, polypeptides, small molecules and metabolites.

REOs as Medicines

Applications

They can be used for a number of applications in this space, including but not limited to the treatment of or application towards: diabetes, oral diseases, gastrointestinal tract diseases, metabolic diseases (e.g., urea cycle disorders, phenylketonuria, hyperammonemia), allergic diseases, autoimmune diseases, prevention of C. difficile infection and diarrheal disorders, diseases associated with dysbiosis, gut inflammation, gastrointestinal inflammation in primary immunodeficiency, irritable bowel diseases (e.g., Crohn's Disease and ulcerative colitis), cardiovascular diseases, liver metastasis, cancer, solid tumors, cancer therapy-associated rashes, progressive glioblastoma, non-small cell lung cancer, HPV-associated cancers, metastatic prostate cancer, hepatic encephalopathy, obesity, diabetes, type 1 diabetes mellitus, P. aeruginosa infection, EHEC/S. aureus/S. epidermis infection, Salmonella infection, Vibrio cholerae infection, oral health of humans and pets, oral mucositis, and novel antibiotics.

Methods of Use

Pharmaceutical compositions comprising the REOs described herein may be used to treat, manage, ameliorate, and/or prevent disease, or symptom(s) associated with disease. Pharmaceutical compositions comprising one or more genetically engineered bacteria, alone or in combination with prophylactic agents, therapeutic agents, and/or pharmaceutically acceptable carriers are provided.

The pharmaceutical compositions of the invention described herein may be formulated in a conventional manner using one or more physiologically acceptable carriers comprising excipients and auxiliaries, which facilitate processing of the active ingredients into compositions for pharmaceutical use. Methods of formulating pharmaceutical compositions are known in the art (e.g., see “Remington's Pharmaceutical Sciences,” Mack Publishing Co., Easton, Pa.). In some embodiments, the pharmaceutical compositions are subjected to tabletting, lyophilizing, direct compression, conventional mixing, dissolving, granulating, levigating, emulsifying, encapsulating, entrapping, or spray drying to form tablets, granulates, nanoparticles, nanocapsules, microcapsules, microtablets, pellets, or powders, which may be enterically coated or uncoated. Appropriate formulation depends on the route of administration.

The REOs may be formulated into pharmaceutical compositions in any suitable dosage form (e.g., liquids, capsules, sachet, hard capsules, soft capsules, tablets, enteric coated tablets, suspension powders, granules, or matrix sustained release formations for oral administration) and for any suitable type of administration (e.g., oral, topical, injectable, intravenous, sub-cutaneous, immediate-release, pulsatile-release, delayed-release, or sustained release). Suitable dosage amounts for the genetically engineered bacteria may range from about 10⁴ to 10¹² bacteria. The composition may be administered once or more daily, weekly, or monthly. The composition may be administered before, during, or following a meal. In one embodiment, the pharmaceutical composition is administered before the subject eats a meal. In one embodiment, the pharmaceutical composition is administered currently with a meal. In on embodiment, the pharmaceutical composition is administered after the subject eats a meal.

The REOs disclosed herein may be administered orally and formulated as tablets, pills, dragees, capsules, liquids, gels, syrups, slurries, suspensions, etc. Pharmacological compositions for oral use can be made using a solid excipient, optionally grinding the resulting mixture, and processing the mixture of granules, after adding suitable auxiliaries if desired, to obtain tablets or dragee cores. Suitable excipients include, but are not limited to, fillers such as sugars, including lactose, sucrose, mannitol, or sorbitol; cellulose compositions such as maize starch, wheat starch, rice starch, potato starch, gelatin, gum tragacanth, methyl cellulose, hydroxypropylmethyl-cellulose, sodium carbomethylcellulose, and/or physiologically acceptable polymers such as polyvinylpyrrolidone (PVP) or polyethylene glycol (PEG). Disintegrating agents may also be added, such as cross-linked polyvinylpyrrolidone, agar, alginic acid or a salt thereof such as sodium alginate.

Liquid preparations for oral administration may take the form of solutions, syrups, suspensions, or a dry product for constitution with water or other suitable vehicle before use. Such liquid preparations may be prepared by conventional means with pharmaceutically acceptable agents such as suspending agents (e.g., sorbitol syrup, cellulose derivatives, or hydrogenated edible fats), emulsifying agents (e.g., lecithin or acacia), non-aqueous vehicles (e.g., almond oil, oily esters, ethyl alcohol, or fractionated vegetable oils), and preservatives (e.g., methyl or propyl-p-hydroxybenzoates or sorbic acid). The preparations may also contain buffer salts, flavoring, coloring, and sweetening agents as appropriate. Preparations for oral administration may be suitably formulated for slow release, controlled release, or sustained release of the genetically engineered microorganisms described herein.

Dosage regimens may be adjusted to provide a therapeutic response. Dosing can depend on several factors, including severity and responsiveness of the disease, route of administration, time course of treatment (days to months to years and time to amelioration of the disease. For example, a single bolus may be administered at one time, several divided doses may be administered over a predetermined period of time, or the dose may be reduced or increased as indicated by the therapeutic situation. The specification for the dosage is dictated by the unique characteristics of the active compound and the particular therapeutic effect to be achieved. Dosage values may vary with the type and severity of the condition to be alleviated. For any particular subject, specific dosage regimens may be adjusted over time according to the individual need and the professional judgment of the treating clinician. Toxicity and therapeutic efficacy of compounds provided herein can be determined by standard pharmaceutical procedures in cell culture or animal models. For example, LD50, ED50, EC50, and IC50 may be determined, and the dose ratio between toxic and therapeutic effects (LD50/ED50) may be calculated as the therapeutic index. Compositions that exhibit toxic side effects may be used, with careful modifications to minimize potential damage to reduce side effects. Dosing may be estimated initially from cell culture assays and animal models. The data obtained from in vitro and in vivo assays and animal studies can be used in formulating a range of dosage for use in humans.

REOs as Research Tools

The use of an REO as a research tool is defined herein as the use of living therapeutics or living vaccines for research or diagnostic purposes. Thus, the use cases above can be modified to include all embodiments that involve analogous scenarios whereby the REO is used similarly but as a research tool rather than a medicine.

REOs as Food Products

Applications

In another embodiment, the composition comprising the REOs of the invention may be a comestible product, for example, a food product. in one embodiment, the food product is milk, concentrated milk, fermented milk (yogurt, sour milk, frozen yogurt, lactic acid bacteria-fermented beverages), milk powder, ice cream, cream cheeses, dry cheeses, soybean milk, fermented soybean milk, vegetable-fruit juices, fruit juices, sports drinks, confectionery, candies, infant foods (such as infant cakes), nutritional food products, animal feeds, or dietary supplements. In one embodiment, the food product is a fermented food, such as a fermented dairy product. In one embodiment, the fermented dairy product is yogurt. In another embodiment, the fermented dairy product is cheese, milk, cream, ice cream, milk shake, or kefir. In another embodiment, the recombinant bacteria of the invention are combined in a preparation containing other live bacterial cells intended to serve as probiotics. In another embodiment, the food product is a beverage. In one embodiment, the beverage is a fruit juice-based beverage or a beverage containing plant or herbal extracts. In another embodiment, the food product is a jelly or a pudding. Other food products suitable for administration of the recombinant bacteria of the invention are well known in the art.

Methods of Use

Methods of use and administration are similar to others methods that have already been referred to herein.

REOs as Environmental Tools

Applications

The REO can be deployed into an open environment to perform a given action. For example, REOs can be used for bioremediation wherein they are used to clean up pollutants at a contaminated site, for example. Examples of contaminated sites can include but are not limited to: soil, water, and subsurface material. Examples of pollutants can include but are not limited to: hydrocarbons, metals, and other toxic waste.

Methods of Use

Methods of use and administration are similar to other methods that have already been referred to herein.

The terms “a” and “an” as used herein mean “one or more” and include the plural unless the context is inappropriate.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

### EXAMPLES

The invention now being generally described, will be more readily understood by reference to the following examples, which are included merely for purposes of illustration of certain aspects and embodiments of the present invention, and is not intended to limit the invention.

### Example 1

### Generation of an RO

An RO is generated from E. coli Nissle 1917 using a the aforementioned recoded genome design, lacking three codons (FIG. 4). The three codons are comprised of one stop codon and two sense codons. This strain is created using methods described previously^{3, 5, 6, 13}, as well as those described or referenced herein. Following recoding, two tRNAs and one release factor are deleted using Lambda Red-mediated homologous recombination.

Upon generation of the RO, codon expansion is performed such that an OTS is electroporated and integrated within the genome of the RO, that incorporates a standard amino acid at forbidden codon 1. Notably, the amino acid incorporated by the OTS at forbidden codon 1 (e.g., amino acid 2) is different than the one previously assigned to forbidden codon 1 (e.g., amino acid 1) prior to recoding and codon expansion.

### Example 2

### Generation of an RRO

This example is designed to produce three RROs from the RO created in Example 1, as medicines for delivery in the gut. One RRO is useful for producing a BP that is a plasmid that may be delivered in the gut, one RRO is useful for producing a BP that is a protein that may be delivered in the gut, and one RRO is useful for producing a BP that is a small molecule that may be delivered in the gut. In this example, these RROs are to be applied as living therapeutics in a human gut application for treatment of a disease, wherein production of a given BP and release of the corresponding RRO into the gut environment generates a therapeutic outcome.

All plasmids and material are made or modified using isothermal assembly and standard cloning. All genomic modifications are made using Lambda Red-mediated homologous recombination either using single stranded DNA oligos or double stranded DNA. The RO contains a mutated mutS gene to enhance retention of desired mutations. All genetic material is introduced using electroporation.

Codon Encryption

Importantly, the exogenous genetic material corresponding to production of the BP is electroporated and in all cases except the plasmid RRO, integrated within the RO's genome. Notably, codon encryption is performed whereby many sites that normally encode amino acid 2 within the transgenic material are replaced with forbidden codon 1, such that the OTS will incorporate amino acid 2 at these forbidden codon 1 sites. Forbidden codons 2 and 3 are left unassigned and serve purely for phage resistance purposes.

Introduction of the Nucleic Acid Sequence Associated with the BP

Plasmid RRO

A plasmid to be amplified, where genes that are only meant to be expressed within the RRO are encrypted and those meant to be expressed outside the RRO are not encrypted, is introduced into the RO by electroporation. The E. coli cells are plated on solid medium containing the antibiotic. Clones are selected and the presence of the plasmid is confirmed by PCR. Clones that contain the plasmid can be used as RROs that produce the plasmid BP, and can be released into an open environment.

Protein RRO

Transgenic material encoding a His-tagged protein product and an antibiotic resistance gene is electroporated into the RO and integrated into the genome. All encoded genes in the transgenic material are encrypted. The E. coli cells are plated on a solid medium containing the antibiotic. Clones are selected and the presence of the transgenic material is confirmed by PCR. Clones that contain the transgenic material can be used as RROs that produce the protein BP, and can be released into an open environment.

Small Molecule RRO

Transgenic material encoding an entire metabolic pathway for the production of the small molecule, and an antibiotic resistance gene is electroporated into the RO and integrated into the genome. All encoded genes in the transgenic material are encrypted. The E. coli cells are plated on a solid medium containing the antibiotic. Clones are selected and the presence of the transgenic material is confirmed by PCR. Clones that contain the transgenic material can be used as RROs that produce the small molecule BP, and can be released into an open environment.

Scaled Down Preliminary Testing of the RRO for BP Production

Following engineering of the RROs, the mutS gene is restored in the final RRO, and Lambda Red genes removed. The three RROs are then assessed by many metrics that include: phage sensitivity, growth in liquid media at microliter scale, growth in liquid media at 2-4 L scale, growth in liquid media at 16 L scale, and production of the desired final BP. Phage sensitivity is tested using assays previously described such as mean lysis time, plaque morphology assessment, and burst size^{6, 37}. The RRO is tested against a panel of phages commonly found in the gut and in bioreactors. Growth in liquid media is assessed by doubling time, max OD600 and overall growth curve assessment. Doubling time is calculated using MATLAB. Production of the desired final BP is tested differently for the three RROs as described below.

Plasmid RRO

Briefly, the RRO is cultured in liquid medium, and grown overnight. The cells are pelleted and lysed, and the plasmid is isolated and purified using a QIAGEN Plasmid Mini or Midi kit. The plasmid yield per gram of cell pellet is assessed using a nanodrop and the quality of the plasmid is assessed by Sanger sequencing and electrophoresis banding patterns.

Protein RRO

Briefly, the RRO is cultured in liquid medium. After the RRO reaches mid-log phase, protein expression is induced and the cells are grown overnight. The cell pellets are collected, lysed, and the His-tagged protein is harvested on nickel resin and eluted with imidazole. The yield per grain of cell pellet and the purity of the protein product are assessed crudely by SDS-PAGE and Coomassie Brilliant Blue staining, and then more specifically by quantifying yield using a Bradford assay. Notably, total protein can also be used as a rough relative comparison before His-tag purification as well, and can be informative.

Small Molecule RRO

Briefly, the RRO is cultured in liquid medium. After the RRO reaches mid-log phase, the metabolic pathway is induced and the cells are grown overnight. The cell pellets are collected, lysed and HPLC and MS are used to detect the small molecule.

### Example 3

### Culturing of RROs

The RROs generated in Example 2, that are capable of biomanufacturing the described BPs, are cultured in a scaled up process similar to that which was used for testing purposes in Example 2, but purely to amplify the RRO in preparation for use in the gut. Processes that are used for culturing, are referenced herein⁵⁰⁻⁵². These processes can occur using cGMP or non cGMP conditions as referenced herein (https://www.fda.gov/drugs/pharmaceutical-quality-resources/current-good-manufacturing-practice-cgmp-regulations).

While bath RROs are expected to be more phage resistant than their cognate base strains, collectively, we expect higher culturing yields of RROs to result from the use of RROs relative to their cognate base strains, especially if phage infection is an existing problem in the facility.

### Example 4

### Uses of RROs

The three different RROs can be cultured as described in Example 3 and separately administered for the therapeutic application. In this case, since these RROs resist both inbound and outbound HGT by phage-dependent and phage-independent mechanisms, they should be safe for use in this open environment without fear that the transgenic material will be shared with native entities in the flora.

### REFERENCES

1 Knappik, A. et al. Fully synthetic human combinatorial antibody libraries (HuCAL) based on modular consensus frameworks and CDRs randomized with trinucleotides. Journal of molecular biology 296, 57-86, doi:10.1006/jmbi.1999.3444 (2000).
2 Rendic, S. & Guengerich, F. P. Survey of Human Oxidoreductases and Cytochrome P450 Enzymes Involved in the Metabolism of Xenobiotic and Natural Chemicals. Chem Res Toxicol 28, 38-42, doi:10.1021/tx500444e (2015).
3 Ostrov, N. et al. Design, synthesis, and testing toward a 57-codon genome. Science 353, 819-822, doi:10.1126/science.aaf3639 (2016).
4 Napolitano, M. G. et al. Emergent rules for codon choice elucidated by editing rare arginine codons in Escherichia coli. Proceedings of the National Academy of Sciences of the United States of America 113, E5588-5597, doi:10.1073/pnas.1605856113 (2016).
5 Isaacs, F. J. et al. Precise manipulation of chromosomes in vivo enables genome-wide codon replacement. Science 333, 348-353 (2011).
6 Lajoie, M. J. et al. Genomically recoded organisms expand biological functions. Science 342, 357-360 (2013).
7 Heinemann, I. U. et al. Enhanced phosphoserine insertion during Escherichia coli protein synthesis via partial UAG codon reassignment and release factor 1 deletion. FEBS letters 586, 3716-3722 (2012).
8 Wannier, T. M. et al. Adaptive evolution of genomically recoded Escherichia coli, Proceedings of the National Academy of Sciences of the United States of America 115, 3090-3095, doi:10.1073/pnas.1715530115 (2018).
9 Kuznetsov, G. et al. Optimizing complex phenotypes through model-guided multiplex genome engineering. Genome biology 18, 100, doi:10.1186/s13059-017-1217-z (2017).
10 Rovner, A. J. et al. Recoded organisms engineered to depend on synthetic amino acids. Nature 518, 89-93, doi:10.1038/nature14095 (2015).
11 Mandell, D. J. et al. Biocontainment of genetically modified organisms by synthetic protein design. Nature 518, 55-60, doi:10.1038/nature14121 (2015).
12 Lajoie, M. J. et al. Probing the limits of genetic recoding in essential genes. Science 342, 361-363 (2013).
13 Fredens, J. et al. Total synthesis of Escherichia coli with a recoded genome. Nature 569, 514-518, doi:10.1038/s41586-019-1192-5 (2019).
14 Amiram, M. et al. Evolution of translation machinery in recoded bacteria enables multi-site incorporation of nonstandard amino acids. Nature biotechnology 33, 1272-1279, doi:10.1038/nbt.3372 (2015).
15 Posfai, G. et al. Emergent properties of reduced-genome Escherichia coli. Science 312, 1044-1046, doi:10.1126/science.1126439 (2006).
16 Kolisnychenko, V. et al. Engineering a reduced Escherichia coli genome. Genome Res 12, 640-647, doi:10.1101/gr.217202 (2002).
17 Umenhoffer, K. et al. Genome-Wide Abolishment of Mobile Genetic Elements Using Genome Shuffling and CRISPR/Cas-Assisted MAGE Allows the Efficient Stabilization of a Bacterial Chassis. ACS Synth Biol. 6, 1471-1483, doi:10.1021/acssynbio.6b00378 (2017).
18 Gibson, D. G. et al. Creation of a bacterial cell controlled by a chemically synthesized genome. Science 329, 52-56 (2010).
19 Hutchison, C. A., 3rd et al. Design and synthesis of a minimal bacterial genome. Science 351, aad6253, doi:10.1126/science.aad6253 (2016).
20 Weinstock, M. T., Hesek, E. D., Wilson, C. M. & Gibson, D. G. Vibrio natriegens as a fast-growing host for molecular biology. Nature methods 13, 849-851, doi:10.1038/nmeth.3970 (2016).
21 Liu, C. C. & Schultz, P. G. Adding new chem is s to the genetic code. Annual review of biochemistry 79, 413-444 (2010).
22 Neumann, H. Rewiring translation—Genetic code expansion and its applications. FEBS letters 586, 2057-2064 (2012).
23 Wang, L., Xie, J. & Schultz, P. G. Expanding the genetic code. Annual review of biophysics and biomolecular structure 35, 225-249 (2006).
24 Xie, J. & Schultz, P. G. A chemical toolkit for proteins—an expanded genetic code. Nature reviews. Molecular cell biology 7, 775-782, doi:10.1038/nrm2005 (2006).
25 Young, T. S. & Schultz, P. G. Beyond the canonical 20 amino acids: expanding the genetic lexicon. The Journal of biological chemistry 285. 11039-11044 (2010).
26 Eggertsson. G. & Soll. D. Transfer ribonucleic acid-mediated suppression of termination codons in Escherichia coli. Microbiological reviews 52, 354-374 (1988).
27 Young, T. S., Ahmad, I., Yin, J. A. & Schultz, P. G. An enhanced system for unnatural amino acid mutagenesis in E. coli. Journal of molecular biology 395, 361-374 (2010).
28 Wang, L, & Schultz, P. G. A general approach for the generation of orthogonal tRNAs, Chemistry & biology 8, 883-890, doi:10.1016/s1074-5521(01)00063-1 (2001).
29 Wang, Y. S. et al. The de novo engineering of pyrrolysyl-tRNA synthetase for genetic incorporation of L-phenylalanine and its derivatives. Molecular bioSystems 7, 714-717 (2011).
30 Arthur, J. C. et al. Intestinal inflammation targets cancer-inducing activity of the microbiota. Science 338, 120-123, doi:10.1126/science.1224820 (2012).
31 Cuevas-Ramos, G. et al. Escherichia coli induces DNA damage in vivo and triggers genomic instability in mammalian cells. Proceedings of the National Academy of Sciences of the United States of America 107, 11537-11542, doi:10.1073/pnas.1001261107 (2010).
32 Olier, M. et al. Genotoxicity of Escherichia coli Nissle 1917 strain cannot be dissociated from its probiotic activity. Gut Microbes 3, 501-509, doi:10.4161/gmic.21737 (2012).
33 Nougayrede, J. P. et al. Escherichia coli induces DNA double-strand breaks in eukaryotic cells. Science 313, 848-851, doi:10.1126/science.1127059 (2006).
34 Mukai, T. et al. Codon reassignment in the Escherichia coli genetic code. Nucleic Acids Res 38, 8188-8195 (2010).
35 Unterholzner, S. J., Poppenberger, B. & Rozhon, W. Toxin-antitoxin systems: Biology, identification, and application. Mob Genet Elements 3, e26219, doi:10.4161/mge.26219 (2013).
36 Bailly-Bechet, M., Vergassola, M. & Rocha, E. Causes for the intriguing presence of tRNAs in phages. Genome Res 17, 1486-1495, doi:10.1101/gr.6649807 (2007).
37 Ma, N. J. & Isaacs, F. J. Genomic. Recoding Broadly Obstructs the Propagation of Horizontally Transferred Genetic Elements. Cell Syst 3, 199-207, doi:10.1016/j.cels.2016.06.009 (2016).
38 Kosuri, S. et al. Scalable gene synthesis by selective amplification of DNA pools from high-fidelity microchips. Nature biotechnology 28, 1295-1299, doi:10.1038/nbt.1716 (2010).
39 Kong. W. et al. Regulated programmed lysis of recombinant Salmonella in host tissues to release protective antigens and confer biological containment. Proceedings of the National Academy of Sciences of the United States of America 105, 9361-9366 (2008).
40 Szafranski, P. et al. A new approach for containment of microorganisms: dual control of streptavidin expression by antisense RNA and the T7 transcription system. Proceedings of the National Academy of Sciences of the United States of America 94, 1059-1063 (1997).
41 Steidler, L. et al. Biological containment of genetically modified Lactococcus lactis for intestinal delivery of human interleukin 10. Nature biotechnology 21, 785-789 (2003).
42 Gallagher, R. R., Patel, J. R., Interiano, A. L., Rovner, A. J. & Isaacs, F. J. Multilayered genetic safeguards limit growth of microorganisms to defined environments. Nucleic Acids Res 43, 1945-1954, doi:10.1093/nar/gku1378 (2015)
43 Wang, H. H. et al. Programming cells by multiplex genome engineering and accelerated evolution. Nature 460, 894-898 (2009).
44 Mosberg, J. A., Lajoie, M. J. & Church, G. M. Lambda Red Recombineering in Escherichia coli Occurs Through a Fully Single-Stranded Intermediate. Genetics 186, 791-U759 (2010).
45 Farzadfard. F. & Lu, T. K. Synthetic biology, Genomically encoded analog memory with precise in vivo DNA writing in living cell populations. Science 346, 1256272, doi:10.1126/science.1256272 (2014).
46 Ellis, H. M., Yu, D., DiTizio, T. & Court, D. L. High efficiency mutagenesis, repair, and engineering of chromosomal DNA using single-stranded oligonucleotides. Proceedings of the National Academy of Sciences of the United States of America 98, 6742-6746 (2001).
47 Sharan, S. K., Thomason, L. C., Kuznetsov, S. G. & Court, D. L. Recombineering: a homologous recombination-based method of genetic engineering. Nature protocols 4, 206-223 (2009).
48 Carr, P. A. et al. Enhanced multiplex genome engineering through co-operative oligonucleotide co-selection. Nucleic Acids Res 40, e132 (2012).
49 Ronda, C., Pedersen, L. E., Sommer, M. O. & Nielsen, A. T. CRMAGE: CRISPR Optimized MAGE Recombineering. Sci Rep 6, 19452, doi:10.1038/srep19452 (2016).
50 Zhang, Y. P., Sun, J. & Ma, Y. Biomanufacturing: history and perspective. J Ind Microbiol Biotechnol 44, 773-784, doi:10.1007/s10295-016-1863-2 (2017).
51 O'Kennedy, R. D., Ward, J. M. & Keshavarz-Moore, E. Effects of fermentation strategy on the characteristics of plasmid DNA production. Biotechnol Appl Biochem 37, 83-90, doi:10.1042/ba20020099 (2003).
52 Xenopoulos, A. & Pattnaik, P. Production and purification of plasmid DNA vaccines: is there scope for further innovation? Expert Rev Vaccines 13, 1537-1551, doi:10.1586/14760584.2014.968556 (2014).

### INCORPORATION BY REFERENCE

The entire disclosure of each of the patent documents and scientific articles referred to herein is incorporated by reference for all purposes.

1. A genetically engineered released bacterial organism comprising engineered genetic material,
the material comprising:
i. a) at least one genetically engineered codon and b) at least one genetically engineered endogenous element, and
ii. at least one exogenous nucleic acid sequence encoding a therapeutic polypeptide or portion thereof,
wherein the at least one genetically engineered naturally occurring element comprises a modification to or deletion of (a) a first nucleic acid sequence encoding a transfer RNA cognate to the genetically engineered codon and optionally (b) a second nucleic acid sequence encoding a release factor cognate to a second genetically engineered second codon.
and wherein the released bacterial organism is capable of producing the therapeutic polypeptide or portion thereof.

2. The genetically engineered released bacterial organism of claim 1, wherein the at least one genetically engineered codon is present within the bacterial genome.

3. The genetically engineered released bacterial organism of claim 1, wherein the at least one genetically engineered codon is present outside the bacterial genome.

4. The genetically engineered released bacterial organism of claim 1, wherein the at least one genetically engineered endogenous element is present within the bacterial genome.

5. The genetically engineered released bacterial organism of claim 1, wherein the at least one genetically engineered endogenous element is present outside the bacterial genome.

6. The genetically engineered released bacterial organism of claim 1, wherein the at least one exogenous nucleic acid sequence is present within the bacterial genome.

7. The genetically engineered released bacterial organism of claim 1, wherein the at least one exogenous nucleic acid sequence is present outside the bacterial genome.

8. The genetically engineered released bacterial organism of claim 1, wherein the engineered genetic material comprises at least one heterologous nucleic acid sequence.

9. The genetically engineered released bacterial organism of claim 1, wherein the engineered genetic material comprises from at least two to over 100 heterologous nucleic acid sequences.

10. The population of claim 1, wherein the engineered genetic material comprises from at least two to over 100 genetically engineered endogenous elements.

11. The genetically engineered released bacterial organism of claim 1, wherein the engineered genetic material comprises synthetic nucleic acid sequences.

12. The genetically engineered released bacterial organism of claim 1, wherein the bacteria comprise Escherichia coli, Escherichia coli NGF-1, Escherichia coli UU2685, Escherichia coli K-12 MG1655, Escherichia coli “recoded” or “GRO” strains and derivatives, Escherichia coli C7 strains, Escherichia coli C7ΔA strains, Escherichia coli C13 strains, Escherichia coli C13ΔA strains, Escherichia coli “C321 strains”, Escherichia coli C321ΔA strains, Escherichia coli C321ΔA “synthetic auxotroph” strains and derivatives, Escherichia coli evolved C321 strains, Escherichia coli C321.ΔA.M9adapted strains, Escherichia coli C321.ΔA.opt strains, Escherichia coli rE.coli-57 strains and derivatives, Escherichia coli C321ΔA “Syn61” strains and derivatives, Escherichia coli K-12 MG1655 “MDS” strains and derivatives, Escherichia coli K-12 MG1655 MDS9 strains, Escherichia coli K-12 MG1655 MDS12 strains, Escherichia coli K-12 MG1655 MDS41 strains, Escherichia coli K-12 MG1655 MDS42 strains, Escherichia coli K-12 MG1655 MDS43 strains, Escherichia coli K-12 MG1655 MDS66 strains, Escherichia coli BL21 DE3, Escherichia coli BL21 hybrid strains (“BLK strains”), Escherichia coli Nissle 1917, Salmonella, Salmonella typhimurium, Salmonella typhi Ty21a, Lactobacillus, Lactobacillus plantarum, Lactobacillus reuteri, Lactobacillus gasseri, Lactobacillus gasseri BNR17, Lactobacillus fermentum KLD, Lactobacillus helveticus, Lactobacillus helveticus strain NS8, Lactococcus, Lactococcus lactis, Lactococcus lactis NZ9000, Lactococcus NZ3900, Lactococcus lactis NZ9001, Lactococcus lactis MG1363, Bacteroides, Bacteroides thetaiotaomicron, Bacteroides fragilis, Bacteroides vulgatus, Bacteroides ovatus, Bacteroides uniformis, Bacteroides eggerthii, Bacteroides xylanisolvens, Bacteroides intestinalis, Bacteroides dorei, Bacteroides cellulosilyticus, Bacillus, Bacillus subtilis, Acetobacter, Streptomyces, Streptococcus, Staphylococcus, Staphylococcus epidermis, Bifidobacterium, Bifidobacterium longum, Bifidobacterium infantis, Eubacterium, Corynebacterium, Corynebacterium glutamicum, Rumunococcus, Coprococcus, Fusobacterium, Clostridium, Clostridium butyricum, Shewanella, Cyanobacterium, Mycoplasma, Mycoplasma capricolum, Mycoplasma genitalium, Mycoplasma mycoides, Mycoplasma mycoides JCVI-syn strains, Mycoplasma mycoides JCVI-syn3.0 strains, Listeria, Listeria monocytogenes, Vibrio, Vibrio cholerae, Vibrio natriegens, Vibrio natriegens Vmax strains, Pseudomonas and variants and progeny thereof

13. The genetically engineered released bacterial organism of claim 1, wherein the at least one genetically engineered codon comprises at least one recoded codon.

14. The genetically engineered released bacterial organism of claim 1, wherein the at least one genetically engineered codon comprises between two and seven recoded codons.

15. The genetically engineered released bacterial organism of claim 1, wherein the at least one genetically engineered codon comprises at least one recoded stop codon.

16. The genetically engineered released bacterial organism of claim 1, wherein the at least one genetically engineered codon comprises at least one recoded sense codon.

17. The genetically engineered released bacterial organism of claim 1, wherein the recoded codon comprises a sense codon, and wherein the recoded codon is synonymously replaced in the engineered genetic material.

18. The genetically engineered released bacterial organism of claim 1, wherein the recoded codon comprises a stop codon, and wherein the recoded codon is synonymously replaced in the engineered genetic material.

19. The genetically engineered released bacterial organism of claim 1, wherein the engineered genetic material comprises a plurality of recoded codons, wherein the recoded codons comprise (i) a sense codon and (ii) a stop codon, and wherein at least one of (i) and (ii) is synonymously replaced in the engineered genetic material.

20. The genetically engineered released bacterial organism of claim 1, wherein the engineered genetic material comprises two to seven recoded codons, wherein the recoded codons comprise (i) a sense codon and (ii) a stop codon, and wherein at least one of (i) and (ii) is synonymously replaced in the engineered genetic material.

21. The genetically engineered released bacterial organism of claim 1, wherein the engineered genetic material comprises replacement of all instances of at least stop codon and at least one sense codon with a second codon in all essential genes.

22. The genetically engineered released bacterial organism of claim 1, wherein the engineered genetic material comprises replacement of all instances of at least stop codon and at least one sense codon with a second codon in all genes essential for viability of the genetically engineered bacterial organism.

23. The genetically engineered released bacterial organism of claim 1, wherein the engineered genetic material comprises replacement of all instances of at least stop codon with a second codon in all genes essential for viability of the genetically engineered bacterial organism.

24. The genetically engineered released bacterial organism of claim 1, wherein the engineered genetic material comprises replacement of all instances of at least one sense codon with a second codon in all genes essential for viability of the genetically engineered bacterial organism.

25. The genetically engineered released bacterial organism of claim 1, wherein the engineered genetic material comprises replacement of all instances of at least stop codon and at least one sense codon with a second codon in all genes essential for bacterial fitness of the genetically engineered bacterial organism.

26. The genetically engineered released bacterial organism of claim 1, wherein the engineered genetic material comprises replacement of all instances of at least stop codon with a second codon in all genes essential for bacterial fitness of the genetically engineered bacterial organism.

27. The genetically engineered released bacterial organism of claim 1, wherein the engineered genetic material comprises replacement of all instances of at least one sense codon with a second codon in all genes essential for bacterial fitness of the genetically engineered bacterial organism.

28. The genetically engineered released bacterial organism of claim 1, wherein the engineered genetic material comprises replacement of all instances of at least stop codon and at least one sense codon with a second codon in all genes essential for bacterial homeostasis of the genetically engineered bacterial organism.

29. The genetically engineered released bacterial organism of claim 1, wherein the engineered genetic material comprises replacement of all instances of at least stop codon with a second codon in all genes essential for bacterial homeostasis of the genetically engineered bacterial organism.

30. The genetically engineered released bacterial organism of claim 1, wherein the engineered genetic material comprises replacement of all instances of at least one sense codon with a second codon in all genes essential for bacterial homeostasis of the genetically engineered bacterial organism.

31. The genetically engineered released bacterial organism of claim 1, wherein the recoded codon comprises a sense codon, and wherein the recoded codon is synonymously replaced in from less than 1% to at least about 99% of the engineered genetic material.

32. The genetically engineered released bacterial organism of claim 1, wherein the recoded codon comprises a stop codon, and wherein recoded codon is synonymously replaced in from less than 1% to at least about 99% of the engineered genetic material.

33. The genetically engineered released bacterial organism of claim 1, comprising a plurality of recoded codons, wherein the recoded codons comprise (i) at least one sense codon and (ii) at least one stop codon, and wherein at least one of (i) and (ii) is synonymously replaced in from less than 1% to at least about 99% of the engineered genetic material.

34. The genetically engineered released bacterial organism of claim 1, wherein the engineered genetic material further comprises at least one orthogonal translation system (OTS) comprising an aminoacyl-tRNA synthetase (aaRS) and cognate tRNA, and wherein the tRNA of the at least one OTS comprises an anticodon complementary to a recoded codon.

35. The genetically engineered released bacterial organism of claim 1, wherein the engineered genetic material further comprises at least one orthogonal translation system (OTS) comprising an aminoacyl-tRNA synthetase (aaRS) and cognate tRNA, wherein the tRNA of the at least one OTS comprises an anticodon complementary to a recoded codon, and wherein the tRNA charges a synthetic or unnatural amino acid.

36. The genetically engineered released bacterial organism of claim 1, wherein the engineered genetic material further comprises at least one orthogonal translation system (OTS) comprising an aminoacyl-tRNA synthetase (aaRS) and cognate tRNA, wherein the tRNA of the at least one OTS comprises an anticodon complementary to a recoded codon, and wherein the tRNA charges a natural amino acid.

37. The genetically engineered released bacterial organism of claim 1, wherein the engineered genetic material further comprises at least one suppressor tRNA, wherein the tRNA of the at least one suppressor tRNA comprises an anticodon complementary to a recoded codon, and wherein the tRNA charges a natural amino acid.

38. The genetically engineered released bacterial organism of claim 1, wherein the engineered genetic material further comprises a deletion or modification to at least one phage receptor gene or portion thereof.

39. The genetically engineered released bacterial organism of claim 1, wherein the engineered genetic material does not comprise a deletion or modification to at least one phage receptor gene or portion thereof.

40. A population comprising a plurality of the genetically engineered released bacterial organism of claim 1, wherein the population is capable of continuously sustaining cGMP manufacturing of the therapeutic polypeptide.

41. The population of claim 40, wherein the population is capable of continuously sustaining cGMP manufacturing of the therapeutic polypeptide in the presence of a phage population.

42. The population of claim 40, wherein the population is capable of continuously sustaining cGMP manufacturing of the therapeutic polypeptide in the presence of an unknown phage population.

43. The population of claim 40, wherein the population has a higher viral resistance capacity compared to a reference bacterial population that comprises the exogenous nucleic acid sequence but does not comprise the at least one genetically engineered codon, and wherein the population is suitable for cGMP manufacturing of the therapeutic polypeptide or a nucleic acid encoding the therapeutic polypeptide.

44. The population of claim 43, wherein the viral resistance capacity allows the population to continuously sustain cGMP manufacturing of the therapeutic polypeptide or a nucleic acid encoding the therapeutic polypeptide in the presence of an unidentified phage population at least about 10% longer than continuously sustained cGMP manufacturing of the therapeutic polypeptide or the nucleic acid encoding the therapeutic polypeptide using the reference bacterial population.

45. The population of claim 43, wherein the viral resistance capacity allows the population to continuously sustain cGMP manufacturing of the therapeutic polypeptide or a nucleic acid encoding the therapeutic polypeptide at least about 10% longer than continuously sustained cGMP manufacturing of the therapeutic polypeptide or the nucleic acid encoding the therapeutic polypeptide using the reference bacterial population.

46. The population of claim 43, wherein the viral resistance capacity allows the population to continuously sustain cGMP manufacturing of the therapeutic polypeptide or a nucleic acid encoding the therapeutic polypeptide from at least about 10% longer to greater than 100% longer than continuously sustained cGMP manufacturing of the therapeutic polypeptide or the nucleic acid encoding the therapeutic polypeptide using the reference bacterial population.

47. The population of claim 43, wherein the viral resistance capacity allows the population to continuously sustain cGMP manufacturing of the therapeutic polypeptide or the nucleic acid encoding the therapeutic polypeptide for greater than 1, 2, 3, 4, 5, 6 or 7 days, or greater than 1, 2, 3, 4 weeks.

48. The population of claim 43, wherein the population has a cGMP manufacturing productivity over a given period of time compared to a reference bacterial population that comprises the exogenous nucleic acid sequence but does not comprise the at least on engineered codon.

49. A genetically engineered released bacterial organism comprising engineered genetic material, the material comprising:
i. a plurality of genetic modifications comprising replacement of all instances of at least one type of first codon with a second codon in all essential genes,
ii. at least one genetically engineered endogenous element, and
iii. at least one exogenous nucleic acid sequence encoding a therapeutic polypeptide or portion thereof,
wherein the at least one genetically engineered endogenous element comprises a modification to or deletion of: (a) a nucleic acid sequence encoding a transfer RNA that recognizes the at least one type of first codon, (b) a nucleic acid sequence encoding a release factor that recognizes the at least one type of first codon, or (c) a combination of (a) and (b) in the same genetically engineered bacterial organism, and
and wherein the released bacterial organism is capable of producing the therapeutic polypeptide or portion thereof.

50. A genetically engineered released bacterial organism comprising engineered genetic material,
the material comprising:
a) at least one genetically engineered codon and b) at least one genetically engineered endogenous element, wherein the at least one genetically engineered endogenous element comprises a modification to or deletion of (a) a first nucleic acid sequence encoding a transfer RNA and optionally (b) a second nucleic acid sequence encoding a release factor,
wherein the released bacterial organism is capable of producing a polypeptide or portion thereof or a nucleic acid.

51. A genetically engineered released bacterial organism comprising engineered genetic material,
the material comprising:
i. a) at least one genetically engineered codon and b) at least one genetically engineered endogenous element, and
ii. at least one exogenous nucleic acid sequence encoding a polypeptide or portion thereof, suitable for synthesis of a therapeutic polypeptide
wherein the at least one genetically engineered naturally occurring element comprises a modification to or deletion of (a) a first nucleic acid sequence encoding a transfer RNA cognate to the genetically engineered codon and optionally (b) a second nucleic acid sequence encoding a release factor cognate to a second genetically engineered second codon.
and wherein the released bacterial organism is capable of producing the therapeutic polypeptide or portion thereof.

52. A genetically engineered released bacterial organism comprising engineered genetic material,
the material comprising:
i. a) at least one genetically engineered codon and b) at least one genetically engineered endogenous element, and
ii. at least one exogenous nucleic acid sequence encoding a polypeptide or portion thereof, suitable for synthesis of a therapeutic nucleic acid
wherein the at least one genetically engineered naturally occurring element comprises a modification to or deletion of (a) a first nucleic acid sequence encoding a transfer RNA cognate to the genetically engineered codon and optionally (b) a second nucleic acid sequence encoding a release factor cognate to a second genetically engineered second codon.
and wherein the released bacterial organism is capable of producing a polypeptide or portion thereof or a nucleic acid.

53. A genetically engineered released bacterial organism comprising engineered genetic material,
the material comprising:
i. a) at least one genetically engineered codon and b) at least one genetically engineered endogenous element, and
ii. at least one exogenous nucleic acid sequence encoding a polypeptide or portion thereof, suitable for synthesis of a therapeutic viral particle
wherein the at least one genetically engineered naturally occurring element comprises a modification to or deletion of (a) a first nucleic acid sequence encoding a transfer RNA cognate to the genetically engineered codon and optionally (b) a second nucleic acid sequence encoding a release factor cognate to a second genetically engineered second codon.
and wherein the released bacterial organism is capable of producing a polypeptide or portion thereof or a nucleic acid.

54. A genetically engineered released bacterial organism comprising engineered genetic material,
the material comprising:
i. a) at least one genetically engineered codon and b) at least one genetically engineered endogenous element, and
ii. at least one exogenous nucleic acid sequence suitable for synthesis of a therapeutic nucleic acid
wherein the at least one genetically engineered naturally occurring element comprises a modification to or deletion of (a) a first nucleic acid sequence encoding a transfer RNA cognate to the genetically engineered codon and optionally (b) a second nucleic acid sequence encoding a release factor cognate to a second genetically engineered second codon.
and wherein the released bacterial organism is capable of producing the therapeutic nucleic acid.

55. A genetically engineered released bacterial organism comprising engineered genetic material,
the material comprising:
i. a) at least one genetically engineered codon and b) at least one genetically engineered endogenous element, and
ii. at least one exogenous nucleic acid sequence encoding a polypeptide or portion thereof, wherein the polypeptide or portion thereof is contacted with a cell ex vivo,
wherein the at least one genetically engineered naturally occurring element comprises a modification to or deletion of (a) a first nucleic acid sequence encoding a transfer RNA cognate to the genetically engineered codon and optionally (b) a second nucleic acid sequence encoding a release factor cognate to a second genetically engineered second codon.
and wherein the released bacterial organism is capable of producing the polypeptide or portion thereof.

56. A genetically engineered released bacterial organism comprising engineered genetic material,
the material comprising:
i. a) at least one genetically engineered codon and b) at least one genetically engineered endogenous element, and
ii. at least one exogenous nucleic acid sequence suitable for synthesis of a nucleic acid
wherein the at least one genetically engineered naturally occurring element comprises a modification to or deletion of (a) a first nucleic acid sequence encoding a transfer RNA cognate to the genetically engineered codon and optionally (b) a second nucleic acid sequence encoding a release factor cognate to a second genetically engineered second codon.
and wherein the released bacterial organism is capable of producing the nucleic acid.

57. A genetically engineered released bacterial organism comprising engineered genetic material,
the material comprising:
i. a) at least one genetically engineered codon and b) at least one genetically engineered endogenous element, and
ii. at least one exogenous nucleic acid sequence suitable for synthesis of a therapeutic nucleic acid, wherein the therapeutic nucleic acid is contacted with a cell ex vivo
wherein the at least one genetically engineered naturally occurring element comprises a modification to or deletion of (a) a first nucleic acid sequence encoding a transfer RNA cognate to the genetically engineered codon and optionally (b) a second nucleic acid sequence encoding a release factor cognate to a second genetically engineered second codon.
and wherein the released bacterial organism is capable of producing the therapeutic nucleic acid.

58. A genetically engineered released bacterial organism comprising engineered genetic material,
the material comprising:
i. a) at least one genetically engineered codon and b) at least one genetically engineered endogenous element, and
ii. at least one exogenous nucleic acid sequence suitable for synthesis of a synthesized nucleic acid, wherein the synthesized nucleic acid is contacted with a cell ex vivo
wherein the at least one genetically engineered naturally occurring element comprises a modification to or deletion of (a) a first nucleic acid sequence encoding a transfer RNA cognate to the genetically engineered codon and optionally (b) a second nucleic acid sequence encoding a release factor cognate to a second genetically engineered second codon.
and wherein the released bacterial organism is capable of producing the synthesized nucleic acid.

59. A genetically engineered released bacterial organism comprising engineered genetic material,
the material comprising:
i. a) at least one genetically engineered codon and b) at least one genetically engineered endogenous element, and
ii. at least one exogenous nucleic acid sequence encoding a polypeptide or portion thereof, suitable for synthesis of a viral particle
wherein the at least one genetically engineered naturally occurring element comprises a modification to or deletion of (a) a first nucleic acid sequence encoding a transfer RNA cognate to the genetically engineered codon and optionally (b) a second nucleic acid sequence encoding a release factor cognate to a second genetically engineered second codon.
and wherein the released bacterial organism is capable of producing the polypeptide or portion thereof.

60. A genetically engineered released bacterial organism comprising engineered genetic material,
the material comprising:
i. a) at least one genetically engineered codon and b) at least one genetically engineered endogenous element, and
ii. at least one exogenous nucleic acid sequence encoding a polypeptide or portion thereof,
wherein the at least one genetically engineered naturally occurring element comprises a modification to or deletion of (a) a first nucleic acid sequence encoding a transfer RNA cognate to the genetically engineered codon and optionally (b) a second nucleic acid sequence encoding a release factor cognate to a second genetically engineered second codon.
and wherein the released bacterial organism is capable of producing the polypeptide or portion thereof.

61. A genetically engineered released bacterial organism comprising engineered genetic material,
the material comprising:
i. a) at least one genetically engineered codon and b) at least one genetically engineered endogenous element, and
ii. at least one exogenous nucleic acid sequence encoding a first polypeptide or portion thereof, suitable for synthesis of a second polypeptide
wherein the at least one genetically engineered naturally occurring element comprises a modification to or deletion of (a) a first nucleic acid sequence encoding a transfer RNA cognate to the genetically engineered codon and optionally (b) a second nucleic acid sequence encoding a release factor cognate to a second genetically engineered second codon.
and wherein the released bacterial organism is capable of producing the first polypeptide or portion thereof.

62. A genetically engineered released bacterial organism comprising engineered genetic material,
the material comprising:
i. a) at least one genetically engineered codon and b) at least one genetically engineered endogenous element, and
ii. at least one exogenous nucleic acid sequence encoding a polypeptide or portion thereof, suitable for synthesis of a nucleic acid
wherein the at least one genetically engineered naturally occurring element comprises a modification to or deletion of (a) a first nucleic acid sequence encoding a transfer RNA cognate to the genetically engineered codon and optionally (b) a second nucleic acid sequence encoding a release factor cognate to a second genetically engineered second codon.
and wherein the released bacterial organism is capable of producing the polypeptide or portion thereof.

63. A method of producing a plasmid, the method comprising culturing the population of genetically engineered released bacteria of any proceeding claim, under conditions such that a plasmid comprising the at least one exogenous nucleic acid sequence is produced.

64. The method of claim 63, wherein the plasmid is produced under cGMP conditions.

65. The method of claim 63, wherein the plasmid is produced in the presence of a phage population.

66. The method of claim 63, wherein the population has resistance to a virus present in the culture, and wherein the culturing comprises a continuous culturing for greater than 1, 2, 3, 4, 5, 6 or 7 days, or greater than 1, 2, 3, 4 weeks.

67. The method of claim 63, wherein the plasmid is capable of generating a virus selected from a lentivirus, adenovirus, herpes virus, adeno-associated virus, or a portion thereof.

68. The method of claim 63, wherein the plasmid is capable of generating a nucleic acid selected from a DNA or an RNA.

69. The method of claim 63, wherein the plasmid is capable of generating an RNA selected from a shRNA, siRNA, mRNA, linear RNA, or circular RNA.

70. A method of producing a polypeptide, the method comprising culturing the population of genetically engineered released bacteria of any proceeding claim, wherein the population comprises at least one exogenous nucleic acid sequence encoding a polypeptide or portion thereof, under conditions such that the polypeptide or portion thereof is produced.

71. The method of claim 70, wherein the polypeptide or portion thereof is produced under cGMP conditions.

72. The method of claim 70, wherein the polypeptide or portion thereof is produced in the presence of a phage population.

73. The method of claim 70, wherein the population has resistance to a virus present in the culture, and wherein the culturing comprises a continuous culturing for greater than 1, 2, 3, 4, 5, 6 or 7 days, or greater than 1, 2, 3, 4 weeks.

74. The method of claim 70, wherein the polypeptide or portion thereof is a human or humanized polypeptide or portion thereof.

75. A method for generating a population of genetically engineered released bacteria, comprising the steps of:
i. contacting an isolated precursor bacterial strain comprising a plurality of bacteria with (i) a first plurality of nucleic acid sequences that replace a first target genome region in the precursor bacterial strain genome, and (ii) a second plurality of nucleic acid sequences that replace a second target genome region in the precursor bacterial strain genome, to produce a genetically engineered bacterium comprising a single nucleic acid sequence from each of the first plurality and the second plurality of nucleic acid sequences;
ii. culturing the genetically engineered bacterium to produce a population of genetically engineered released bacteria.

76. The method of claim 75, wherein each of the first plurality and the second plurality of nucleic acid sequences comprise at least one genetically engineered endogenous element comprises a modification to or deletion of (a) a first nucleic acid sequence encoding a transfer RNA and optionally (b) a second nucleic acid sequence encoding a release factor.
